SEQUENCE LISTING (179 sequences)

SEQ ID NO: 1 - DNA, 1805 bp, Homo sapiens
  source 1..1805: mol_type = genomic DNA
agtagcagtagcagtagcagtagcagtagcagtagcagtagcagtagcagcagcagaaccagaatttccccgcacgtgtctcaggctctcatttgccaactcagtctctaagtatttttattggcaggaaaaataaaatagctatgagtgaaataattcattagacctgagcctccatcaattttgtgtttaaaggcctgactctctttacctttccctgggatggaagatgcaaatgttcctgatgtcactgtcaaaaaagaagaaccagtgggtatattgtatgcttgagttccagccatttgtcacaatagatagagatgactgccatgtgtgtagactttctatagactgtgtgctaaacccgacctgccacttccaaggagtagatgaggaatgtccatggttctggggagccctaccccaatttggggcagacattccaaagctcattttctgtggagggggttgatggttaaaggacggcctgggagtaactcgtctgtactagggcccaggagagttacatgctgcttcccatgttattcatcattcccccatgtgaatagctatggcgtgaggtccaaggttagggcctttctaccataaatgggggaataaaattcccctaccagcctgagaagtttctgttataaagaggcctttttttttgcgggggtgggggagcaagccgactaatgtgttatttccatacggtttgttttaaaatgtagacgtcatatgcaggagaggtggtgtagtgagtcacaacgggattagaaggaccagtccgaaaagcagaagagggtcaagttcagggcacggaggactactgcattcagtggcgtgaaaggcagatggctgaacaggagggggacattacattgcttgttctccttgagcctcgatttcctcatctaaaaagagggtcatttattcacagaacatttattaaacttgtgccaggcaccgtgccaggagctggactaaaaattaaatccacccctgtgagctgctctgaaggctaaaatatgaagtatgtaaaagtaaccaagtgctgtacacatgcagctattcaatgactgtgtgggcattgcggcagattttaattttcttttttatttctttctctttagtgagaggtgttggttgttattattgtcgtcgctgtaactgtctatttcacttgcttttttgttgcctccagcccattccagggctgtcatctaggacacttcttatcacctaaataaccggggaggcaaagcgctttcttaagagatggatccagaagaacaatgctggttttctgtacaaaaaggggctgtgggaagtagagataagaagggaattggccaagatgaatgtacagagccttattttttttataacacagcaagattagatacaaaacaggacaatagcatcatctgtttttataactggaaaggacctcactttacaggtggggaagaatagagtggagaagtgaagagaatggtcacagagtcaatcagcatgtctgcgtcaaagctgggattcccgattcagggctcttactacagtgacgtatggctaatattttggcattgtttcggggaaaagctgaagccctgatggtgtacgtcactcttgagatagtctgtagtccagcagggaggaaagcaaggaagggaggtggaggcagcatttttgggtgtaacatttcgttcttgttttgtggccaaatcatagtgtgattgggacaagccactgcctttctctgagcctccactttctttttcttcttaagagggagggaatag

SEQ ID NO: 2 - DNA, 753 bp, Mus musculus
  source 1..753: mol_type = genomic DNA
ttgtcgcgacaaatagagatgaccgccaagtgtgcagacattccacagacgcatagtcccgtaatgcagctcctaaggagtgggtgaggaatgtaagcagtgcaggaaagacctactgcttgctttgtggggatggtcaatcactggatagaggtcagtttgtgaataaagcatcagttcatgagaccaggggagcagtacgttcattcccgatgttactactaatctgtccatatgaacagtcccgctatgagggccaaggcaaggactcgctgttggaatgaaagcccctcaaccagcctggacttttcctggtgttttgggggatggggtaactagtatattatttccacacactgttttaaaagatagttatcttacccaagagatgtatgggggtgagtcagcttggggccagaggaatcagtctcccaagaaaaggagggcaaagttcagggcgcagaggatgaccatattcttaaggtaaaaggagatagctagccaggaggtagaagttatcttgtaagttctttctgggtttcaatttcctcatccaaaggtgtcaccacatatttattattatgttcttctcctaggcaccaagcccagatgctgggctgaaaaatcaaactcattcctataaactgttctgagggtaacacatgacttaacgcaaaatgtacagtatagtacttttgttgcttctaggccatgtttggtctgtgatctgggacacttcttgccacttaaata

SEQ ID NO: 3 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cacctctcctgcatatgacg

SEQ ID NO: 4 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cctctcctgcatatgacgtc

SEQ ID NO: 5 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cttccatcccagggaaaggt

SEQ ID NO: 6 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gcttttcggactggtccttc

SEQ ID NO: 7 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctgctgctgctactgctact

SEQ ID NO: 8 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ttgcatcttccatcccaggg

SEQ ID NO: 9 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tctggttctgctgctgctac

SEQ ID NO: 10 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ccacctctcctgcatatgac

SEQ ID NO: 11 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gcagctcacaggggtggatt

SEQ ID NO: 12 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
agcaatgtaatgtccccctc

SEQ ID NO: 13 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
aaatctgccgcaatgcccac

SEQ ID NO: 14 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ggttctgctgctgctactgc

SEQ ID NO: 15 - DNA, 20 bp, synthetic construct
  source 1..20: mol_type = other DNA
  misc_feature 1..5: note = RNA
  misc_feature 6..15: note = DNA
  misc_feature 16..20: allele = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tcttggccaattcccttctt

SEQ ID NO: 16 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
acctctcctgcatatgacgt

SEQ ID NO: 17 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gagactgagttggcaaatga

SEQ ID NO: 18 - DNA, 20 bp, synthetic construct
  source 1..20: mol_type = other DNA
  misc_feature 1..5: note = RNA
  misc_feature 6..15: note = DNA
  misc_feature 16..20: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
acagacgagttactcccagg

SEQ ID NO: 19 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctgctgctactgctactgct

SEQ ID NO: 20 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
catcaaccccctccacagaa

SEQ ID NO: 21 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tcttccatcccagggaaagg

SEQ ID NO: 22 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tgctgctgctactgctactg

SEQ ID NO: 23 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tggccaattcccttcttatc

SEQ ID NO: 24 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ttcatcttggccaattccct

SEQ ID NO: 25 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tggatccatctcttaagaaa

SEQ ID NO: 26 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
atcttggccaattcccttct

SEQ ID NO: 27 - DNA, 20 bp, synthetic construct
  source 1..20: mol_type = other DNA
  misc_feature 1..5: note = RNA
  misc_feature 6..15: note = DNA
  misc_feature 16..20: standard_name = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctattccctccctcttaaga

SEQ ID NO: 28 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cccgcaaaaaaaaaggcctc

SEQ ID NO: 29 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ttctggttctgctgctgcta

SEQ ID NO: 30 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ggggaaattctggttctgct

SEQ ID NO: 31 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cggggaaattctggttctgc

SEQ ID NO: 32 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gcaaatgagagcctgagaca

SEQ ID NO: 33 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
actgagttggcaaatgagag

SEQ ID NO: 34 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tgccaataaaaatacttaga

SEQ ID NO: 35 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctgccaataaaaatacttag

SEQ ID NO: 36 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
atagctattttatttttcct

SEQ ID NO: 37 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
atttcactcatagctatttt

SEQ ID NO: 38 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctaatgaattatttcactca

SEQ ID NO: 39 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tctaatgaattatttcactc

SEQ ID NO: 40 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
taaacacaaaattgatggag

SEQ ID NO: 41 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ttaaacacaaaattgatgga

SEQ ID NO: 42 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tttaaacacaaaattgatgg

SEQ ID NO: 43 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cccagggaaaggtaaagaga

SEQ ID NO: 44 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tttgcatcttccatcccagg

SEQ ID NO: 45 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
caagcatacaatatacccac

SEQ ID NO: 46 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
atggctggaactcaagcata

SEQ ID NO: 47 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
aatggctggaactcaagcat

SEQ ID NO: 48 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cgaggctcaaggagaacaag

SEQ ID NO: 49 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ccgcaatgcccacacagtca

SEQ ID NO: 50 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tacagcgacgacaataataa

SEQ ID NO: 51 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gttcttctggatccatctct

SEQ ID NO: 52 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
tccacctcccttccttgctt

SEQ ID NO: 53 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
aaagtggaggctcagagaaa

SEQ ID NO: 54 - DNA, 16 bp, synthetic construct
  source 1..16: mol_type = other DNA
  misc_feature 1..3: note = RNA
  modified_base 1..3: mod_base = OTHER; note = 2'-methoxyethyl (2'-MOE) modified nucleoside
  misc_feature 4..13: note = DNA
  misc_feature 14..16: note = RNA
  modified_base 14..16: mod_base = OTHER; note = 2'-methoxyethyl (2'-MOE) modified nucleoside
tctacacacatggcag

SEQ ID NO: 55 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gtctacacacatggca

SEQ ID NO: 56 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
agtctacacacatggc

SEQ ID NO: 57 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
aagtctacacacatgg

SEQ ID NO: 58 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tctatagaaagtctac

SEQ ID NO: 59 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gtctatagaaagtcta

SEQ ID NO: 60 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
agtctatagaaagtct

SEQ ID NO: 61 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
acagtctatagaaagt

SEQ ID NO: 62 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
cacagtctatagaaag

SEQ ID NO: 63 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
aagtggcaggtcgggt

SEQ ID NO: 64 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gcggggaaattctggt

SEQ ID NO: 65 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tgcggggaaattctgg

SEQ ID NO: 66 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gtgcggggaaattctg

SEQ ID NO: 67 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
cgtgcggggaaattct

SEQ ID NO: 68 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
acacgtgcggggaaat

SEQ ID NO: 69 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gacacgtgcggggaaa

SEQ ID NO: 70 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tcgggtttagcacaca

SEQ ID NO: 71 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ggtcgggtttagcaca

SEQ ID NO: 72 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
aggtcgggtttagcac

SEQ ID NO: 73 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gcaggtcgggtttagc

SEQ ID NO: 74 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ggcaggtcgggtttag

SEQ ID NO: 75 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tggcaggtcgggttta

SEQ ID NO: 76 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
caaattggggtagggc

SEQ ID NO: 77 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gccgtcctttaaccat

SEQ ID NO: 78 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tactcccaggccgtcc

SEQ ID NO: 79 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ttactcccaggccgtc

SEQ ID NO: 80 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
cgagttactcccaggc

SEQ ID NO: 81 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
acgagttactcccagg

SEQ ID NO: 82 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gacgagttactcccag

SEQ ID NO: 83 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
agacgagttactccca

SEQ ID NO: 84 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
cagacgagttactccc

SEQ ID NO: 85 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
acagacgagttactcc

SEQ ID NO: 86 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ccctagtacagacgag

SEQ ID NO: 87 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gccctagtacagacga

SEQ ID NO: 88 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ggccctagtacagacg

SEQ ID NO: 89 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gggccctagtacagac

SEQ ID NO: 90 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tgggccctagtacaga

SEQ ID NO: 91 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ctgggccctagtacag

SEQ ID NO: 92 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
cctgggccctagtaca

SEQ ID NO: 93 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tcctgggccctagtac

SEQ ID NO: 94 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gtagaaaggccctaac

SEQ ID NO: 95 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tcggcttgctccccca

SEQ ID NO: 96 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gtcggcttgctccccc

SEQ ID NO: 97 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ttagtcggcttgctcc

SEQ ID NO: 98 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
attagtcggcttgctc

SEQ ID NO: 99 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gactcactacaccacc

SEQ ID NO: 100 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
aatcccgttgtgactc

SEQ ID NO: 101 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ctaatcccgttgtgac

SEQ ID NO: 102 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ccttctaatcccgttg

SEQ ID NO: 103 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gtccttctaatcccgt

SEQ ID NO: 104 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gcttttcggactggtc

SEQ ID NO: 105 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tgcttttcggactggt

SEQ ID NO: 106 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ctgcttttcggactgg

SEQ ID NO: 107 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tccgtgccctgaactt

SEQ ID NO: 108 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ctccgtgccctgaact

SEQ ID NO: 109 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
agtagtcctccgtgcc

SEQ ID NO: 110 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gcagtagtcctccgtg

SEQ ID NO: 111 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tgcagtagtcctccgt

SEQ ID NO: 112 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
catctgcctttcacgc

SEQ ID NO: 113 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ccgcaatgcccacaca

SEQ ID NO: 114 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
atctgccgcaatgccc

SEQ ID NO: 115 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
aatctgccgcaatgcc

SEQ ID NO: 116 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gttacagcgacgacaa

SEQ ID NO: 117 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
agttacagcgacgaca

SEQ ID NO: 118 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
cagttacagcgacgac

SEQ ID NO: 119 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
agacagttacagcgac

SEQ ID NO: 120 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ccccggttatttaggt

SEQ ID NO: 121 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tccccggttatttagg

SEQ ID NO: 122 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ctccccggttatttag

SEQ ID NO: 123 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
tgagacacgtgcgggg

SEQ ID NO: 124 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ctgagacacgtgcggg

SEQ ID NO: 125 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
cctgagacacgtgcgg

SEQ ID NO: 126 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
agtggcaggtcgggtt

SEQ ID NO: 127 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
gaagtggcaggtcggg

SEQ ID NO: 128 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
ctactccttggaagtg

SEQ ID NO: 129 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
aaattggggtagggct

SEQ ID NO: 130 - DNA, 16 bp, synthetic construct
  source 1..16: mol_type = other DNA
  misc_feature 1..3: note = RNA
  modified_base 1..3: mod_base = OTHER; note = locked nucleic acid (LNA) modified nucleoside (cMe; beta-D-methyleneoxy)
  misc_feature 4..13: note = DNA
  misc_feature 14..16: note = RNA
  modified_base 14..16: mod_base = OTHER; note = locked nucleic acid (LNA) modified nucleoside (cMe; beta-D-methyleneoxy)
aggtaaagagagtcag

SEQ ID NO: 131 - DNA, 15 bp, synthetic construct
  source 1..15: mol_type = other DNA
  misc_feature 1..3: note = RNA
  misc_feature 4..12: note = DNA
  misc_feature 13..15: note = RNA
  modified_base 1..3: mod_base = OTHER; note = 2'-methoxyethyl (2'-MOE) modified nucleoside
  modified_base 13..15: mod_base = OTHER; note = 2'-methoxyethyl (2'-MOE) modified nucleoside
aacacgtctatacgc

SEQ ID NO: 132 - DNA, 15 bp, synthetic construct
  source 1..15: mol_type = other DNA
  misc_feature 1..3: note = RNA
  misc_feature 4..12: note = DNA
  misc_feature 13..15: note = RNA
  modified_base 1..3: mod_base = OTHER; note = locked nucleic acid (LNA) modified nucleoside (cMe; beta-D-methyleneoxy)
  modified_base 13..15: mod_base = OTHER; note = locked nucleic acid (LNA) modified nucleoside (cMe; beta-D-methyleneoxy)
aacacgtctatacgc

SEQ ID NO: 133 - DNA, 20 bp, synthetic construct
  source 1..20: mol_type = other DNA
  misc_feature 1..5: note = RNA
  misc_feature 16..20: note = RNA
  misc_feature 6..15: note = DNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
gacacgtgcggggaaattct

SEQ ID NO: 134 - DNA, 20 bp, synthetic construct
  source 1..20: mol_type = other DNA
  misc_feature 1..6: note = RNA
  misc_feature 15..20: note = RNA
  misc_feature 7..14: note = DNA
  modified_base 1..6: mod_base = OTHER; note = 2'-methoxyethyl (2'-MOE) modified nucleoside
  modified_base 15..20: mod_base = OTHER; note = 2'-methoxyethyl (2'-MOE) modified nucleoside
gacacgtgcggggaaattct

SEQ ID NO: 135 - DNA, 20 bp, synthetic construct
  source 1..20: mol_type = other DNA
  misc_feature 1..7: note = RNA
  misc_feature 14..20: note = RNA
  misc_feature 8..13: note = DNA
  modified_base 1..7: mod_base = OTHER; note = 2'-methoxyethyl (2'-MOE) modified nucleoside
  modified_base 14..20: mod_base = OTHER; note = 2'-methoxyethyl (2'-MOE) modified nucleoside
gacacgtgcggggaaattct

SEQ ID NO: 136 - DNA, 18 bp, synthetic construct
  source 1..18: mol_type = other DNA
  misc_feature 1..4: note = RNA
  misc_feature 5..14: note = DNA
  misc_feature 15..18: note = RNA
  modified_base 1..4: mod_base = OTHER; note = 2'-methoxyethyl (2'-MOE) modified nucleoside
  modified_base 15..18: mod_base = OTHER; note = 2'-methoxyethyl (2'-MOE) modified nucleoside
gacacgtgcggggaaatt

SEQ ID NO: 137 - DNA, 18 bp, synthetic construct
  source 1..18: mol_type = other DNA
  misc_feature 1..5: note = RNA
  misc_feature 6..13: note = DNA
  misc_feature 14..18: note = RNA
  modified_base 1..5: mod_base = OTHER; note = 2'-methoxyethyl (2'-MOE) modified nucleoside
  modified_base 14..18: mod_base = OTHER; note = 2'-methoxyethyl (2'-MOE) modified nucleoside
gacacgtgcggggaaatt

SEQ ID NO: 138 - DNA, 16 bp, synthetic construct
  source 1..16: mol_type = other DNA
  misc_feature 1..4: note = RNA
  misc_feature 5..12: note = DNA
  misc_feature 13..16: note = RNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
gacacgtgcggggaaa

SEQ ID NO: 139 - DNA, 16 bp, synthetic construct
  source 1..16: mol_type = other DNA
  misc_feature 1..5: note = RNA
  misc_feature 6..11: note = DNA
  misc_feature 12..16: note = RNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
gacacgtgcggggaaa

SEQ ID NO: 140 - DNA, 17 bp, synthetic construct
  source 1..17: mol_type = other DNA
  misc_feature 1..3: note = RNA
  misc_feature 14..17: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
gacacgtgcggggaaat

SEQ ID NO: 141 - DNA, 17 bp, synthetic construct
  source 1..17: mol_type = other DNA
  misc_feature 1..5: note = RNA
  misc_feature 6..13: note = DNA
  misc_feature 14..17: note = RNA
  modified_base 1..5: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
gacacgtgcggggaaat

SEQ ID NO: 142 - DNA, 16 bp, synthetic construct
  source 1..16: mol_type = other DNA
  misc_feature 1..5: note = RNA
  misc_feature 6..12: note = DNA
  misc_feature 13..16: note = RNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
gacacgtgcggggaaa

SEQ ID NO: 143 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  modified_base 6: mod_base = OTHER; note = 2'OMe modified deoxynucleoside
gacacgtgcggggaaa

SEQ ID NO: 144 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
acagacgagttactcccagg

SEQ ID NO: 145 - DNA, 20 bp, synthetic construct
  source 1..20: mol_type = other DNA
  modified_base 1..6: mod_base = OTHER; note = 2'-methoxyethyl (2'-MOE) modified nucleoside
  misc_feature 1..6: note = RNA
  misc_feature 7..14: note = DNA
  modified_base 15..20: mod_base = OTHER; note = 2'-methoxyethyl (2'-MOE) modified nucleoside
  misc_feature 15..20: note = RNA
acagacgagttactcccagg

SEQ ID NO: 146 - DNA, 20 bp, synthetic construct
  source 1..20: mol_type = other DNA
  misc_feature 1..7: note = RNA
  misc_feature 8..13: note = DNA
  misc_feature 14..20: note = RNA
  modified_base 1..7: mod_base = OTHER; note = 2'-methoxyethyl (2'-MOE) modified nucleoside
  modified_base 14..20: mod_base = OTHER; note = 2'-methoxyethyl (2'-MOE) modified nucleoside
acagacgagttactcccagg

SEQ ID NO: 147 - DNA, 18 bp, synthetic construct
  source 1..18: mol_type = other DNA
  misc_feature 1..4: note = RNA
  misc_feature 5..14: note = DNA
  misc_feature 15..18: note = RNA
  modified_base 1..4: mod_base = OTHER; note = 2'-methoxyethyl (2'-MOE) modified nucleoside
  modified_base 15..18: mod_base = OTHER; note = 2'-methoxyethyl (2'-MOE) modified nucleoside
acagacgagttactccca

SEQ ID NO: 148 - DNA, 18 bp, synthetic construct
  source 1..18: mol_type = other DNA
  misc_feature 1..5: note = RNA
  misc_feature 6..13: note = DNA
  misc_feature 14..18: note = RNA
  modified_base 1..5: mod_base = OTHER; note = 2'-methoxyethyl (2'-MOE) modified nucleoside
  modified_base 14..18: mod_base = OTHER; note = 2'-methoxyethyl (2'-MOE) modified nucleoside
acagacgagttactccca

SEQ ID NO: 149 - DNA, 16 bp, synthetic construct
  source 1..16: mol_type = other DNA
  misc_feature 1..4: note = RNA
  misc_feature 5..12: note = DNA
  misc_feature 13..16: note = RNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
cagacgagttactccc

SEQ ID NO: 150 - DNA, 16 bp, synthetic construct
  source 1..16: mol_type = other DNA
  misc_feature 1..5: note = RNA
  misc_feature 6..11: note = DNA
  misc_feature 12..16: note = RNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
cagacgagttactccc

SEQ ID NO: 151 - DNA, 17 bp, synthetic construct
  source 1..17: mol_type = other DNA
  misc_feature 1..3: note = RNA
  misc_feature 14..17: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
acagacgagttactccc

SEQ ID NO: 152 - DNA, 17 bp, synthetic construct
  source 1..17: mol_type = other DNA
  misc_feature 1..5: note = RNA
  misc_feature 6..13: note = DNA
  misc_feature 14..17: note = RNA
  modified_base 1..5: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
acagacgagttactccc

SEQ ID NO: 153 - DNA, 16 bp, synthetic construct
  source 1..16: mol_type = other DNA
  misc_feature 1..5: note = RNA
  misc_feature 6..12: note = DNA
  misc_feature 13..16: note = RNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
cagacgagttactccc

SEQ ID NO: 154 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  modified_base 6: mod_base = OTHER; note = 2'OMe modified deoxynucleoside
cagacgagttactccc

SEQ ID NO: 155 - DNA, 20 bp, synthetic construct
  source 1..20: mol_type = other DNA
  modified_base 1..6: mod_base = OTHER; note = 2'-methoxyethyl (2'-MOE) modified nucleoside
  misc_feature 1..6: note = RNA
  misc_feature 7..14: note = DNA
  modified_base 15..20: mod_base = OTHER; note = 2'-methoxyethyl (2'-MOE) modified nucleoside
  misc_feature 15..20: note = RNA
gttcttctggatccatctct

SEQ ID NO: 156 - DNA, 20 bp, synthetic construct
  source 1..20: mol_type = other DNA
  misc_feature 1..7: note = RNA
  misc_feature 8..13: note = DNA
  misc_feature 14..20: note = RNA
  modified_base 1..7: mod_base = OTHER; note = 2'-methoxyethyl (2'-MOE) modified nucleoside
  modified_base 14..20: mod_base = OTHER; note = 2'-methoxyethyl (2'-MOE) modified nucleoside
gttcttctggatccatctct

SEQ ID NO: 157 - DNA, 18 bp, synthetic construct
  source 1..18: mol_type = other DNA
  misc_feature 1..4: note = RNA
  misc_feature 5..14: note = DNA
  misc_feature 15..18: note = RNA
  modified_base 1..4: mod_base = OTHER; note = 2'-methoxyethyl (2'-MOE) modified nucleoside
  modified_base 15..18: mod_base = OTHER; note = 2'-methoxyethyl (2'-MOE) modified nucleoside
gttcttctggatccatct

SEQ ID NO: 158 - DNA, 18 bp, synthetic construct
  source 1..18: mol_type = other DNA
  misc_feature 1..5: note = RNA
  misc_feature 6..13: note = DNA
  misc_feature 14..18: note = RNA
  modified_base 1..5: mod_base = OTHER; note = 2'-methoxyethyl (2'-MOE) modified nucleoside
  modified_base 14..18: mod_base = OTHER; note = 2'-methoxyethyl (2'-MOE) modified nucleoside
gttcttctggatccatct

SEQ ID NO: 159 - DNA, 16 bp, synthetic construct
  source 1..16: mol_type = other DNA
  misc_feature 1..4: note = RNA
  misc_feature 5..12: note = DNA
  misc_feature 13..16: note = RNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
gttcttctggatccat

SEQ ID NO: 160 - DNA, 16 bp, synthetic construct
  source 1..16: mol_type = other DNA
  misc_feature 1..5: note = RNA
  misc_feature 6..11: note = DNA
  misc_feature 12..16: note = RNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
gttcttctggatccat

SEQ ID NO: 161 - DNA, 17 bp, synthetic construct
  source 1..17: mol_type = other DNA
  misc_feature 1..3: note = RNA
  misc_feature 14..17: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
gttcttctggatccatc

SEQ ID NO: 162 - DNA, 17 bp, synthetic construct
  source 1..17: mol_type = other DNA
  misc_feature 1..5: note = RNA
  misc_feature 6..13: note = DNA
  misc_feature 14..17: note = RNA
  modified_base 1..5: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
gttcttctggatccatc

SEQ ID NO: 163 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
gttcttctggatccat

SEQ ID NO: 164 - DNA, 16 bp, synthetic construct
  source 1..16: mol_type = other DNA
  misc_feature 1..5: note = RNA
  misc_feature 6..12: note = DNA
  misc_feature 13..16: note = RNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
gttcttctggatccat

SEQ ID NO: 165 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  modified_base 6: mod_base = OTHER; note = 2'OMe modified deoxynucleoside
gttcttctggatccat

SEQ ID NO: 166 - DNA, 20 bp, synthetic construct
  source 1..20: mol_type = other DNA
  modified_base 1..6: mod_base = OTHER; note = 2'-methoxyethyl (2'-MOE) modified nucleoside
  misc_feature 1..6: note = RNA
  misc_feature 7..14: note = DNA
  modified_base 15..20: mod_base = OTHER; note = 2'-methoxyethyl (2'-MOE) modified nucleoside
  misc_feature 15..20: note = RNA
tacagcgacgacaataataa

SEQ ID NO: 167 - DNA, 20 bp, synthetic construct
  source 1..20: mol_type = other DNA
  misc_feature 1..7: note = RNA
  misc_feature 8..13: note = DNA
  misc_feature 14..20: note = RNA
  modified_base 1..7: mod_base = OTHER; note = 2'-methoxyethyl (2'-MOE) modified nucleoside
  modified_base 14..20: mod_base = OTHER; note = 2'-methoxyethyl (2'-MOE) modified nucleoside
tacagcgacgacaataataa

SEQ ID NO: 168 - DNA, 18 bp, synthetic construct
  source 1..18: mol_type = other DNA
  misc_feature 1..4: note = RNA
  misc_feature 5..14: note = DNA
  misc_feature 15..18: note = RNA
  modified_base 1..4: mod_base = OTHER; note = 2'-methoxyethyl (2'-MOE) modified nucleoside
  modified_base 15..18: mod_base = OTHER; note = 2'-methoxyethyl (2'-MOE) modified nucleoside
tacagcgacgacaataat

SEQ ID NO: 169 - DNA, 18 bp, synthetic construct
  source 1..18: mol_type = other DNA
  misc_feature 1..5: note = RNA
  misc_feature 6..13: note = DNA
  misc_feature 14..18: note = RNA
  modified_base 1..5: mod_base = OTHER; note = 2'-methoxyethyl (2'-MOE) modified nucleoside
  modified_base 14..18: mod_base = OTHER; note = 2'-methoxyethyl (2'-MOE) modified nucleoside
tacagcgacgacaataat

SEQ ID NO: 170 - DNA, 16 bp, synthetic construct
  source 1..16: mol_type = other DNA
  misc_feature 1..4: note = RNA
  misc_feature 5..12: note = DNA
  misc_feature 13..16: note = RNA
  modified_base 1..4: mod_base = OTHER; note = 2'-methoxyethyl (2'-MOE) modified nucleoside
  modified_base 13..16: mod_base = OTHER; note = 2'-methoxyethyl (2'-MOE) modified nucleoside
tacagcgacgacaata

SEQ ID NO: 171 - DNA, 16 bp, synthetic construct
  source 1..16: mol_type = other DNA
  misc_feature 1..5: note = RNA
  misc_feature 6..11: note = DNA
  misc_feature 12..16: note = RNA
  modified_base 1..5: mod_base = OTHER; note = 2'-methoxyethyl (2'-MOE) modified nucleoside
  modified_base 12..16: mod_base = OTHER; note = 2'-methoxyethyl (2'-MOE) modified nucleoside
tacagcgacgacaata

SEQ ID NO: 172 - DNA, 17 bp, synthetic construct
  source 1..17: mol_type = other DNA
  misc_feature 1..3: note = RNA
  misc_feature 14..17: note = RNA
  misc_feature 4..13: note = DNA
  modified_base 1..3: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
tacagcgacgacaataa

SEQ ID NO: 173 - DNA, 17 bp, synthetic construct
  source 1..17: mol_type = other DNA
  misc_feature 1..5: note = RNA
  misc_feature 6..13: note = DNA
  misc_feature 14..17: note = RNA
  modified_base 1..5: mod_base = OTHER; note = locked nucleic acid (LNA) modified nucleoside (cMe; beta-D-methyleneoxy)
  modified_base 14..17: mod_base = OTHER; note = locked nucleic acid (LNA) modified nucleoside (cMe; beta-D-methyleneoxy)
tacagcgacgacaataa

SEQ ID NO: 174 - DNA, 16 bp, synthetic construct
  source 1..16: mol_type = other DNA
  misc_feature 1..3: note = RNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
tacagcgacgacaata

SEQ ID NO: 175 - DNA, 16 bp, synthetic construct
  source 1..16: mol_type = other DNA
  misc_feature 1..5: note = RNA
  misc_feature 6..12: note = DNA
  misc_feature 13..16: note = RNA
  modified_base 1..5: mod_base = OTHER; note = locked nucleic acid (LNA) modified nucleoside (cMe; beta-D-methyleneoxy)
  modified_base 13..16: mod_base = OTHER; note = locked nucleic acid (LNA) modified nucleoside (cMe; beta-D-methyleneoxy)
tacagcgacgacaata

SEQ ID NO: 176 - DNA, 16 bp, synthetic construct
  source 1..16: mol_type = other DNA
  misc_feature 1..3: note = DNA
  misc_feature 4..13: note = DNA
  misc_feature 14..16: note = RNA
  modified_base 1..3: mod_base = OTHER; note = locked nucleic acid (LNA) modified nucleoside (cMe; beta-D-methyleneoxy)
  modified_base 14..16: mod_base = OTHER; note = locked nucleic acid (LNA) modified nucleoside (cMe; beta-D-methyleneoxy)
  modified_base 6: mod_base = OTHER; note = 2'OMe modified deoxynucleoside
tacagcgacgacaata

SEQ ID NO: 177 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cgccctgaactttgccctcc

SEQ ID NO: 178 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
cacacttggcggtcatctct

SEQ ID NO: 179 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-methoxyethyl (2'-MOE) modified nucleoside
  modified_base 16..20: mod_base = OTHER; note = 2'-methoxyethyl (2'-MOE) modified nucleoside
ctcacccccatacatctctt

(19) United States
(12) Patent Application Publication
(10) Pub. No.: US 2025/0115914 A1
(43) Pub. Date: Apr. 10, 2025

(54) COMPOUNDS FOR INHIBITION OF IL-1BETA EXPRESSION

(71) Applicant: Lemba BV, Nijmegen (NL)

(72) Inventors: Makiri Kawasaki, Tokyo (JP); Stephanie Berry, Scarsdale, NY (US); Ezio Fok, Nijmegen (NL)

(21) Appl. No.: 18/906,483

(22) Filed: Oct. 4, 2024

Related U.S. Application Data
(60) Provisional application No. 63/542,514, filed on Oct. 4, 2023.

(51) Int. Cl. C12N 15/113

(52) U.S. Cl. CPC C12N 15/1136; C12N 2310/11; C12N 2310/321; C12N 2310/3231; C12N 2310/341

(57) ABSTRACT

There is disclosed a gapmer type of ASO (antisense oligonucleotide) and pharmaceutical compositions comprising gapmers, wherein the gapmer compounds specifically inhibit IL-1β eRNA transcription and wherein the gapmers comprise from about 14 to about 25 nucleotide bases having (3′ to 5′) a 3′ wing region having from 3 to 7 chemically modified RNA bases, a gap region comprising from at least 8 DNA bases, and a 5′ wing region having from 3 to 7 chemically modified RNA bases, and wherein the gapmer is substantially complementary to a 14-25 base region on IL-1β eRNA (SEQ ID NO. 1). Preferably, the gapmer nucleotides are each linked by phosphorothioate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

### PRIOR RELATED APPLICATIONS

This application claims priority to U.S. Provisional Application Ser. No. 63/542,514, filed Oct. 4, 2023, the contents of which are herein incorporated in their entirety.

### SEQUENCE STATEMENT

The instant application contains a Sequence Listing, which has been submitted electronically and is hereby incorporated by reference in its entirety. Said file is named J1742-00102_SL SEQ.xml and is about 555 kb in size.

### FIELD

The present disclosure provides a gapmer type of ASOs (antisense oligonucleotides) and pharmaceutical compositions comprising gapmers, wherein the gapmer compounds specifically inhibit IL-1β-eRNA transcription and wherein the gapmers comprise from about 14 to about 25 nucleotide bases having (3′ to 5′) a 3′ wing region having from 3 to 7 chemically modified RNA bases, a gap region comprising from at least 8 DNA bases, and a 5′ wing region having from 3 to 7 chemically modified RNA bases, and wherein the gapmer is substantially complementary to a 14-25 base region on IL-1β eRNA (SEQ ID NO. 1). Preferably, the gapmer nucleotides are each linked by phosphorothiolate (P═S) internucleotide bonds throughout the Gapmer; and wherein the modified nucleotide base modifications are selected from the group consisting of 2′-methoxyethyl (MOE) nucleotides, locked nucleic acid nucleotides (LNA), and combinations thereof. The present disclosure further provides a method for treating sepsis comprising administering to a septic individual an effective amount of a pharmaceutical composition comprising a gapmer, wherein the gapmer compound specifically inhibit IL-1β eRNA transcription and wherein the gapmer comprises from about 14 to about 25 nucleotide bases having (3′ to 5′) a 3′ wing region having from 3 to 7 chemically modified RNA bases, a gap region comprising from at least 8 DNA bases, and a 5′ wing region having from 3 to 7 chemically modified RNA bases, and wherein the gapmer is substantially complementary to a 14-25 base region on IL-1β eRNA (SEQ ID NO. 1).

### BACKGROUND

The following includes information that may be useful in understanding the present invention. It is not an admission that any of the information, publications or documents specifically or implicitly referenced herein is prior art, or important, to the inventions described and claimed herein. All publications, patents, related applications, and other written or electronic materials mentioned or identified herein are hereby incorporated herein by reference in their entirety. The information incorporated is as much a part of the application as filed as if all of the text and all other content was repeated in the application and should be treated as part of the text and content of the application as filed.

Long non-coding RNAs (lncRNAs) are non-coding transcripts of more typically than 200 nucleotides (200 nt). LncRNAs (long non-coding RNAs) emanating from enhancers are often called enhancer RNAs (eRNAs). One therapeutic target area is IL-1β-eRNA, which is an eRNA that regulates the transcriptional activation of IL-1β. Therefore, transcription inhibitors or regulators of IL-1β eRNA are needed to address both acute and chronic inflammation. Mattick, J. S., et al. Nat Rev Mol Cell Biol 24, 430-447 (2023)). Long non-coding transcripts are found in many species. DNA (cDNA) sequencing projects such as FANTOM have identified the complexity of these transcripts in humans (Carninci P, et al. (September 2005), Science. 309 (5740): 1559-1563).

Interleukin-1 beta (IL-1β) is a master cytokine positioned at the apex of inflammation, serves as a key endogenous mediator of trained immunity. It induces powerful pro-inflammatory functions that are crucial for the induction of trained immunity and providing immunological protection (Arts, R. J. W. et al. BCG vaccination protects against experimental viral infection in humans through the induction of cytokines associated with trained immunity. Cell Host Microbe 23, 89-100. e5 (2018)). Transcription of IL-1β eRNA (enhancer RNA) is needed to elicit a potent IL-1β-mediated inflammatory response. However, overproduction of IL-1β-eRNA can be detrimental and contribute to many hyper-inflammatory and autoimmune diseases, such as sepsis, rheumatoid arthritis, gout, and other auto-inflammatory syndromes (Dinarello, C. A. A clinical perspective of IL-1β as the gatekeeper of inflammation. Eur. J. Immunol. 41, 1203-1217 (2011)). Therefore, transcription inhibitors or regulators of IL-1β-eRNA are needed to address both acute and chronic inflammation.

LncRNAs (long non-coding RNAs) emanating from enhancers are often referred to as enhancer RNAs (eRNAs). One therapeutic target area is IL-1β-eRNA, which is an eRNA that regulates the transcriptional activation of IL-1β (Ilott, N. E. et al. Long non-coding RNAs and enhancer RNAs regulate the lipopolysaccharide-induced inflammatory response in human monocytes. Nat. Commun. 5, 3979 (2014)). Therefore, transcription inhibitors or 35 regulators of IL-1β eRNA are needed to address both acute and chronic inflammation.

### SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

The present disclosure provides a gapmer type of ASOs (antisense oligonucleotides) and pharmaceutical compositions comprising gapmers, wherein the gapmer compounds specifically inhibit IL-1β eRNA transcription and wherein the gapmers comprise from about 14 to about 25 nucleotide bases having (3′ to 5′) a 3′ wing region having from 3 to 7 chemically modified RNA bases, a gap region comprising from at least 8 DNA bases, and a 5′ wing region having from 3 to 7 chemically modified RNA bases, and wherein the gapmer is substantially complementary to a 14-25 base region on IL-1β0 eRNA (SEQ ID NO. 1). Preferably, the gapmer nucleotides are each linked by phosphorothiolate (P═S) internucleotide bonds throughout the gapmer; and wherein the modified nucleotide base modifications are selected from the group consisting of 2′-methoxyethyl (MOE) nucleotides, locked nucleic acid nucleotides (LNA), and combinations thereof.

The present disclosure provides a gapmer compound that is complementary to Region A of IL-1β eRNA (SEQ ID NO. 1 bases 58 to 80), and that inhibits multiple acute inflammatory gene transcription regulated by IL-1β eRNA, comprising: (a) a 5′ wing sequence having from about 3 to about 7 wing modified nucleotide bases; (b) a central gap region sequence having from about 8 to about 15 2′ deoxynucleotides; and (c) a 3′ wing sequence having from about 3 to about 7 wing modified nucleotide bases; wherein the gapmer nucleotides are each linked by phosphorothioate internucleoside linkages, phosphorothiolate internucleoside linkages, or combinations thereof throughout the gapmer; and wherein the modified nucleotide base modifications are selected from the group consisting of 2′-methoxyethyl (MOE) nucleotides, locked nucleic acid nucleotides (LNA), and combinations thereof. Preferably, Region A of IL-1β eRNA is SEQ ID NO. 1 base 65-80. Most preferably the gapmer compound that is complementary to Region A is selected from the group consisting of SEQ ID NOs. 64, 65, 68, 69, and combinations thereof.

The present disclosure provides a gapmer compound that is complementary to Region B of IL-1β eRNA (SEQ ID NO. 1 bases 1153 to 1172), and that inhibits multiple acute inflammatory gene transcription regulated by IL-1β eRNA, comprising: (a) a 5′ wing sequence having from about 3 to about 7 wing modified nucleotide bases; (b) a central gap region sequence having from about 8 to about 15 2′ deoxynucleotides; and (c) a 3′ wing sequence having from about 3 to about 7 wing modified nucleotide bases; wherein the gapmer nucleotides are each linked by phosphorothioate internucleoside linkages, phosphorothiolate internucleoside linkages, or combinations thereof throughout the gapmer; and wherein the modified nucleotide base modifications are selected from the group consisting of 2′-methoxyethyl (MOE) nucleotides, locked nucleic acid nucleotides (LNA), and combinations thereof. Preferably, the gapmer compound that is complementary to Region B is selected from the group consisting of SEQ ID NOs. 50, 174, and combinations thereof.

The present disclosure provides a gapmer compound that is complementary to Region C of IL-1β eRNA (SEQ ID NO. 1 bases 1245 to 1297), and that inhibits multiple acute inflammatory gene transcription regulated by IL-1β eRNA, comprising: (a) a 5′ wing sequence having from about 3 to about 7 wing modified nucleotide bases; (b) a central gap region sequence having from about 8 to about 15 2′ deoxynucleotides; and (c) a 3′ wing sequence having from about 3 to about 7 wing modified nucleotide bases; wherein the gapmer nucleotides are each linked by phosphorothioate internucleoside linkages, phosphorothiolate internucleoside linkages, or combinations thereof throughout the gapmer; and wherein the modified nucleotide base modifications are selected from the group consisting of 2′-methoxyethyl (MOE) nucleotides, locked nucleic acid nucleotides (LNA), and combinations thereof. Preferably, Region C of IL-1β0 eRNA is SEQ ID NO. 1 base 1246-1261. More preferably, the gapmer compound that is complementary to Region B is selected from the group consisting of SEQ ID NOs. 25, 51, 121, 122, and combinations thereof.

### DETAILED DISCLOSURE

The inventions relate to IL-1β eRNA transcriptional modulators and compositions comprising thereof for the modulation of IL-1β eRNA transcription. In some embodiments, the IL-1β eRNA transcriptional modulators are IL-1β eRNA transcriptional inhibitors.

In some embodiments, the IL-1β eRNA transcriptional inhibitors comprises an antisense molecule. See Examples, below.

A first series of gapmer compounds of the present disclosure were designed to target different regions of the human IL-1 long non-coding RNA IL-1β eRNA. (FANTOM CAT number: FTMT20800006485.1) (SEQ ID NO. 1).

### Human IL-1β-eRNA Sequence:

AGTAGCAGTAGCAGTAGCAGTAGCAGTAGCAGTAGCAGTAGCAGTAGCA
GCAGCAGAACCAGAATTTCCCCGCACGTGTCTCAGGCTCTCATTTGCCA
ACTCAGTCTCTAAGTATTTTTATTGGCAGGAAAAATAAAATAGCTATGA
GTGAAATAATTCATTAGACCTGAGCCTCCATCAATTTTGTGTTTAAAGG
CCTGACTCTCTTTACCTTTCCCTGGGATGGAAGATGCAAATGTTCCTGA
TGTCACTGTCAAAAAAGAAGAACCAGTGGGTATATTGTATGCTTGAGTT
CCAGCCATTTGTCACAATAGATAGAGATGACTGCCATGTGTGTAGACTT
TCTATAGACTGTGTGCTAAACCCGACCTGCCACTTCCAAGGAGTAGATG
AGGAATGTCCATGGTTCTGGGGAGCCCTACCCCAATTTGGGGCAGACAT
TCCAAAGCTCATTTTCTGTGGAGGGGGTTGATGGTTAAAGGACGGCCTG
GGAGTAACTCGTCTGTACTAGGGCCCAGGAGAGTTACATGCTGCTTCCC
ATGTTATTCATCATTCCCCCATGTGAATAGCTATGGCGTGAGGTCCAAG
GTTAGGGCCTTTCTACCATAAATGGGGGAATAAAATTCCCCTACCAGCC
TGAGAAGTTTCTGTTATAAAGAGGCCTTTTTTTTTGCGGGGGTGGGGGA
GCAAGCCGACTAATGTGTTATTTCCATACGGTTTGTTTTAAAATGTAGA
CGTCATATGCAGGAGAGGTGGTGTAGTGAGTCACAACGGGATTAGAAGG
ACCAGTCCGAAAAGCAGAAGAGGGTCAAGTTCAGGGCACGGAGGACTAC
TGCATTCAGTGGCGTGAAAGGCAGATGGCTGAACAGGAGGGGGACATTA
CATTGCTTGTTCTCCTTGAGCCTCGATTTCCTCATCTAAAAAGAGGGTC
ATTTATTCACAGAACATTTATTAAACTTGTGCCAGGCACCGTGCCAGGA
GCTGGACTAAAAATTAAATCCACCCCTGTGAGCTGCTCTGAAGGCTAAA
ATATGAAGTATGTAAAAGTAACCAAGTGCTGTACACATGCAGCTATTCA
ATGACTGTGTGGGCATTGCGGCAGATTTTAATTTTCTTTTTTATTTCTT
TCTCTTTAGTGAGAGGTGTTGGTTGTTATTATTGTCGTCGCTGTAACTG
TCTATTTCACTTGCTTTTTTGTTGCCTCCAGCCCATTCCAGGGCTGTCA
TCTAGGACACTTCTTATCACCTAAATAACCGGGGAGGCAAAGCGCTTTC
TTAAGAGATGGATCCAGAAGAACAATGCTGGTTTTCTGTACAAAAAGGG
GCTGTGGGAAGTAGAGATAAGAAGGGAATTGGCCAAGATGAATGTACAG
AGCCTTATTTTTTTTATAACACAGCAAGATTAGATACAAAACAGGACAA
TAGCATCATCTGTTTTTATAACTGGAAAGGACCTCACTTTACAGGTGGG
GAAGAATAGAGTGGAGAAGTGAAGAGAATGGTCACAGAGTCAATCAGCA
TGTCTGCGTCAAAGCTGGGATTCCCGATTCAGGGCTCTTACTACAGTGA
CGTATGGCTAATATTTTGGCATTGTTTCGGGGAAAAGCTGAAGCCCTGA
TGGTGTACGTCACTCTTGAGATAGTCTGTAGTCCAGCAGGGAGGAAAGC
AAGGAAGGGAGGTGGAGGCAGCATTTTTGGGTGTAACATTTCGTTCTTG
TTTTGTGGCCAAATCATAGTGTGATTGGGACAAGCCACTGCCTTTCTCT
GAGCCTCCACTTTCTTTTTCTTCTTAAGAGGGAGGGAATAG

Corresponding Mouse IL1β_eRNA, or Also Called IL-1β eRNA Sequence (SEQ ID NO. 2):

TTGTCGCGACAAATAGAGATGACCGCCAAGTGTGCAGACATTCCACAGA
CGCATAGTCCCGTAATGCAGCTCCTAAGGAGTGGGTGAGGAATGTAAGC
AGTGCAGGAAAGACCTACTGCTTGCTTTGTGGGGATGGTCAATCACTGG
ATAGAGGTCAGTTTGTGAATAAAGCATCAGTTCATGAGACCAGGGGAGC
AGTACGTTCATTCCCGATGTTACTACTAATCTGTCCATATGAACAGTCC
CGCTATGAGGGCCAAGGCAAGGACTCGCTGTTGGAATGAAAGCCCCTCA
ACCAGCCTGGACTTTTCCTGGTGTTTTGGGGGATGGGGTAACTAGTATA
TTATTTCCACACACTGTTTTAAAAGATAGTTATCTTACCCAAGAGATGT
ATGGGGGTGAGTCAGCTTGGGGCCAGAGGAATCAGTCTCCCAAGAAAAG
GAGGGCAAAGTTCAGGGCGCAGAGGATGACCATATTCTTAAGGTAAAAG
GAGATAGCTAGCCAGGAGGTAGAAGTTATCTTGTAAGTTCTTTCTGGGT
TTCAATTTCCTCATCCAAAGGTGTCACCACATATTTATTATTATGTTCT
TCTCCTAGGCACCAAGCCCAGATGCTGGGCTGAAAAATCAAACTCATTC
CTATAAACTGTTCTGAGGGTAACACATGACTTAACGCAAAATGTACAGT
ATAGTACTTTTGTTGCTTCTAGGCCATGTTTGGTCTGTGATCTGGGACA
CTTCTTGCCACTTAAATA.

### Definitions

The following terms have the following meanings:

“2′-substituted nucleoside” means a nucleoside comprising a 2′-substituted sugar moiety. “2′-substituted” in reference to a sugar moiety means a sugar moiety comprising at least one 2′-substituent group other than H or OH.

“2′-deoxynucleoside” means a nucleoside comprising 2′-H furanosyl sugar moiety, as found naturally occurring in deoxyribonucleosides (DNA). A 2′-deoxynucleoside may comprise a modified nucleobase or may comprise an RNA nucleobase (e.g., uracil).

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methyl cytosine” means a cytosine modified with a methyl group attached to a 5 position. A 5-methyl cytosine is a modified nucleobase.

“About” means plus or minus 7% of the provided value.

“Active pharmaceutical agent” means the substance or substances in a pharmaceutical composition that provide a therapeutic benefit when administered to an individual. For example, in certain embodiments an antisense oligonucleotide targeted to IL-1β-eRNA is an active pharmaceutical agent.

“Active target region” or “target region” means a region to which one or more active antisense compounds is targeted. “Active antisense compounds” means antisense compounds that reduce target gene transcription or resulting protein levels.

“Administering” means providing a pharmaceutical agent to an individual, and includes, but is not limited to administering by a medical professional and self-administering.

As used herein, the term “IL-1β-eRNA transcription modulator” gapmertype ASO compound (also sometimes referred to as an IL-1β-eRNA transcription “inhibitor” gapmer type ASO compound) is a compound that prevents, inhibits, and/or reduces the transcription IL-1β-eRNA.

“Inhibits” or “modulates” should not be taken to imply that the function, activity, expression, trafficking and/or assembly of IL-1β-eRNA is completely inhibited or deactivated or wholly modulated, although this may be preferred, but should be taken to include any reduction in the function, activity, or expression, of IL-1β-eRNA.

“Amelioration” refers to a lessening of at least one indicator, sign, or symptom of an associated disease, disorder, or condition. The severity of indicators may be determined by subjective or objective measures, which are known to those skilled in the art.

“Antisense activity” means any detectable and/or measurable change attributable to the hybridization of an antisense compound to its target nucleic acid. Antisense activity is a decrease in the amount or expression of a target nucleic acid or protein encoded by such target nucleic acid compared to target nucleic acid levels or target protein levels in the absence of the antisense compound.

“Antisense compound” means an oligomeric compound capable of achieving at least one antisense activity.

“alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo; cycloaliphatic [e.g., cycloalkyl or cycloalkenyl]; heterocycloaliphatic [e.g., heterocycloalkyl or heterocycloalkenyl]; aryl; heteroaryl; alkoxy; aroyl; heteroaroyl; acyl [e.g., (aliphatic)carbonyl, (cycloaliphatic)carbonyl, or (heterocycloaliphatic)carbonyl]; nitro; cyano; amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl]; amino [e.g., aliphaticamino, cycloaliphaticamino, or heterocycloaliphaticamino]; sulfonyl [e.g., aliphatic-S(O)₂—]; sulfinyl; sulfanyl; sulfoxy; urea; thiourea; sulfamoyl; sulfamide; oxo; carboxy; carbamoyl; cycloaliphaticoxy; heterocycloaliphaticoxy; aryloxy; heteroaryloxy; aralkyloxy; heteroarylalkoxy; alkoxycarbonyl; alkylcarbonyloxy; or hydroxy. Without limitation, some examples of substituted alkyls include carboxyalkyl (such as HOOC-alkyl, alkoxycarbonylalkyl, and alkylcarbonyloxyalkyl); cyanoalkyl; hydroxyalkyl; alkoxyalkyl; acylalkyl; aralkyl; (alkoxyaryl)alkyl; (sulfonylamino)alkyl (such as alkyl-S(O)₂-aminoalkyl); aminoalkyl; amidoalkyl; (cycloaliphatic)alkyl; or haloalkyl.

“alkylene” refers to a bifunctional alkyl group.

A “bifunctional” moiety refers to a chemical group that is attached to the main chemical structure in two places, such as a linker moiety. Bifunctional moieties can be attached to the main chemical structure at any two chemically feasible substitutable points. Unless otherwise specified, bifunctional moieties can be in either direction, e.g. the bifunctional moiety “N—O” can be attached in the —N—O— direction or the —O—N— direction.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition or may be in separate pharmaceutical compositions. Each of the two or more pharmaceutical agents may be administered through the same or different routes of administration. Co-administration encompasses parallel or sequential administration.

“Complementarity” means the capacity for pairing between nucleobases of a first nucleic acid and a second nucleic acid.

“Contiguous nucleobases” means nucleobases immediately adjacent to each other. “Diluent” means an ingredient in a composition that lacks pharmacological activity, but is pharmaceutically necessary or desirable. For example, the diluent in an injected composition may be a liquid, e.g. saline solution.

“Dose” means a specified quantity of a pharmaceutical agent provided in a single administration, or in a specified time-period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period-of-time or continuously. Doses may be stated as the amount of pharmaceutical agent per hour, day, week, or month.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid. “Substantially complementary” means that within an oligonucleotide, there are at most two mismatch bases which are not complementary to a second nucleic acid. For example, for a 10-mer oligonucleotide, a sequence which is substantially complementary can have 8, 9, or 10 bases which are complementary to a second nucleic acid. “Substantially complementary” includes “fully complementary” sequences (where there are no bases which are mismatched within the oligonucleotide sequence to a second nucleic acid.

“Chemically Modified” when referring to RNA or DNA bases, has its meaning understood in the art and includes a nucleoside base selected from the group consisting of 2′-substituted nucleoside, ‘—O-methoxyethyl” (also 2′-MOE and 2′-O(CH₂)₂—OCH₃), 2′-deoxynucleoside, 2′-O-methoxyethyl nucleotide, 5-methyl cytosine, monocylic nucleosides, Bicyclic nucleoside, 4′-2′ bicyclic nucleoside, 4′ to 2′ bicyclic nucleoside, locked nucleic acid, and Nucleoside mimetic, all as defined herein.

“Cleavable moiety” means a bond or group of atoms that is cleaved under physiological conditions, for example, inside a cell or an animal.

“Complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another.

Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. “Fully complementary” or “100% complementary” in reference to oligonucleotides means that oligonucleotides are complementary to another oligonucleotide or nucleic acid at each nucleoside of the oligonucleotide.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Gapmer” means a modified oligonucleotide comprising an internal “gap” region having a plurality of DNA nucleosides positioned between external regions having one or more nucleosides, wherein the nucleosides comprising the internal region are chemically distinct from the nucleoside or nucleosides comprising the external regions. The internal region is often referred to as the “gap” and the external regions is often referred to as the “wings.” Unless otherwise indicated, “gapmer” refers to a sugar motif. Unless otherwise indicated, the sugar moieties of the nucleosides of the gap central region of a gapmer are unmodified 2′-deoxyribosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications.

“Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

“Immediately adjacent” means there are no intervening elements between the immediately adjacent elements.

“Individual” or “Subject” used interchangeably herein, means a human or non-human animal selected for treatment or therapy.

“Modified nucleotide base” and “modified nucleoside” refers to a deoxyribose nucleotide or ribose nucleotide that is modified to have one or more chemical moieties not found in the natural nucleic acids. Examples of modified nucleotide bases, and “modified nucleosides” are compounds of Formula Ia, Formula Ib, Formula IIa, or Formula IIb as described herein.

A “Non-bicyclic modified sugar moiety” refers to the sugar moiety of a modified nucleotide base, as described herein, wherein the chemical modifications do not involve the transformation of the sugar moiety into a bicyclic or multicyclic ring system.

“Monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

“2′-modified sugar” means a furanosyl sugar modified at the 2′ position. Such modifications include substituents as described herein.

“Bicyclic nucleoside” (BNA) refers to a modified nucleoside comprising a bicyclic sugar moiety. Examples of bicyclic nucleosides include without limitation nucleosides comprising a bridge between the 4′ and the 2′ ribosyl ring atoms. The synthesis of bicyclic nucleosides have been disclosed in, for example, 7,399,845, WO/2009/006478, WO/2008/150729, US2004-0171570, U.S. Pat. No. 7,427,672, Chattopadhyaya et al. J. Org. Chem. 2009, 74, 118-134, WO 99/14226, and WO 2008/154401. The synthesis and preparation of the methyleneoxy (4′-CH₂— O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and their preparation are also described in WO98/39352 and WO 99/14226. Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported. One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

A “4′-2′ bicyclic nucleoside” or “4′ to 2′ bicyclic nucleoside” is a bicyclic nucleoside comprising a furanose ring comprising a bridge connecting two carbon atoms of the furanose ring connects the 2′ carbon atom and the 4′ carbon atom of the sugar ring.

A “locked nucleic acid” (LNA) is a modified nucleotide base, wherein the chemical modifications are transformation of the sugar moiety into a bicyclic or multicyclic ring system. Two specific examples of locked nucleic acid compounds are β-D-methyleneoxy nucleotides, or “constrained methyl” (cMe) nucleotides; and β-D-ethyleneoxy nucleotides, or “constrained ethyl” (cEt) nucleotides.

“Mismatch” or “non-complementary” means a nucleobase of a first oligonucleotide that is not complementary with the corresponding nucleobase of a second oligonucleotide or target nucleic acid when the first and second oligonucleotide are aligned.

“Motif” means the pattern of unmodified and/or modified sugar moieties, nucleobases, and/or internucleoside linkages, in an oligonucleotide.

“Nucleobase” means an unmodified nucleobase or a modified nucleobase. An “unmodified nucleobase” is adenine (A), thymine (T), cytosine (C), uracil (U), and guanine (G). A “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. “Nucleobase sequence” means the order of contiguous nucleobases in a nucleic acid or oligonucleotide independent of any sugar or internucleoside linkage modification.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Pharmaceutically acceptable carriers” means physiologically and pharmaceutically acceptable carriers of compounds. Pharmaceutically acceptable carriers retain the desired biological activity of the parent compound and do not impart undesired toxicological effects thereto.

“Pharmaceutical composition” means a mixture of substances suitable for administering to an animal. For example, a pharmaceutical composition may comprise an oligomeric compound and a sterile aqueous solution. In certain embodiments, a pharmaceutical composition shows activity in free uptake assay in certain cell lines.

“Pharmaceutically acceptable derivative” encompasses pharmaceutically acceptable carriers, conjugates, prodrugs or isomers of the compounds described herein.

“Phosphorothioate linkage” means a linkage between nucleosides where the phosphodiester bond is modified by replacing one of the non-bridging oxygen atoms with a sulfur atom.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2-H(H) deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

“Targeting” or “targeted” means the process of design and selection of an antisense compound that will specifically hybridize to a target nucleic acid and induce a desired effect.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Target nucleic acid” and “target RNA” mean a nucleic acid that an antisense compound is designed to affect, such as IL-1β-eRNA.

“Target region” means a portion of a target nucleic acid to which an oligomeric compound is designed to hybridize.

“Therapeutically effective amount” means an amount of a pharmaceutical agent that provides a therapeutic benefit to an individual.

“Treat” refers to administering a pharmaceutical composition to effect an alteration or improvement of a disease, disorder, or condition.

“Unmodified nucleotide” means a nucleotide composed of naturally occurring nucleobases, sugar moieties, and internucleoside linkages. An unmodified nucleotide is an RNA nucleotide (i.e. β-D-ribonucleosides) or a DNA nucleotide (i.e. β-D-deoxyribonucleoside).

### Antisense ASO Compound Motifs

The present disclosure provides a gapmer antisense oligonucleotide (ASO) compound (“gapmer compound”) that is substantially, including 100% complementary over the entire length of the gapmer compound) to a region of IL-1β eRNA, and that inhibits multiple acute inflammatory gene transcription regulated by the IL-1β-eRNA. By “substantially complementary”, it means no more than two mismatches over the entire length of the gapmer. In some embodiments, there is one mismatch between the gapmer compound and the target region on the IL-1β eRNA. In some embodiments, there are two mismatches between the gapmer compound and the target region on the IL-1β-eRNA. In some embodiments, a gapmer compound comprises a modified oligonucleotide of 12 to 29 linked nucleosides in length. The gapmer compound is substantially complementary (for example, having no more than one nucleotide mismatch over the entire length of the gapmer compound) to a region of IL-1β eRNA (SEQ ID NO. 1), and inhibits multiple acute inflammatory gene transcription from being regulated by the IL-1β-eRNA.

A gapmer includes an internal region has a plurality of nucleotides or linked nucleosides that are positioned between external regions having a plurality of nucleotides or linked nucleosides that are chemically distinct from the nucleotides or linked nucleosides of the internal region. In the case of an antisense oligonucleotide having a gapmer motif, the gap segment generally serves as the substrate for endonuclease cleavage, while the wing segments comprise modified nucleosides. The regions of a gapmer (5′ wing, gap sequence, and 3′ wing) are differentiated by the types of sugar moieties comprising each distinct region. The types of sugar moieties that are used to differentiate the regions of a gapmer may include β-D-ribonucleosides, β-D-deoxyribonucleosides, 2′-modified nucleosides (such 2′-modified nucleosides may include 2′-MOE, and 2′-O—CH3, among others), and bicyclic sugar modified nucleosides (such bicyclic sugar modified nucleosides may include those having a 4′-(CH2)n-O-2′ bridge, where n=1 or n=2). Preferably, each distinct region comprises uniform sugar moieties. The wing-gap-wing motif is “X-Y-Z”, where “X” represents the length of the 5′ wing region, “Y” represents the length of the gap region, and “Z” represents the length of the 3′ wing region. In general, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Often, X and Z are the same chemistry of modified nucleobase, or they are different. Preferably, Y is between 8 and 15 nucleotides. X or Z can be any of 3, 4, 5, 6, 7, 8, 9, or 10 nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6 or 5-8-5.

In one embodiment, a gapmer has a gap segment of ten 2′-deoxyribonucleotides positioned immediately adjacent to and between wing segments of five chemically modified nucleosides. In certain embodiments, the chemical modification in the wings comprises a 2′-sugar modification. In another embodiment, the chemical modification comprises a 2′-MOE sugar modification. Preferably, a gap-widened antisense oligonucleotide has a gap segment of eight 2′-deoxyribonucleotides positioned immediately adjacent to and between wing segments of five chemically modified nucleosides. Or the chemical modification comprises a 2′-sugar modification. Or the chemical modification comprises a 2′-MOE sugar modification.

A gapmer has a gap segment of eight 2′-deoxyribonucleotides positioned immediately adjacent to and between wing segments of five to six chemically modified nucleosides. The chemical modification comprises a 2′-sugar modification, such as a 2′-MOE sugar modification. The gapmer compounds having a nucleotide sequence over its entire length that is substantially complementary to the nucleotide sequence of SEQ ID NO: 1, for example, to one of the Regions A-C described herein, including the entire length of Region B of IL-1β eRNA, nucleotides 1153-1172, of Region A of IL-1β eRNA nucleotides 58-83 of SEQ ID NO: 1, and Region C of IL-1β eRNA nucleotides 1245-1297 of SEQ ID NO: 1.

The chemical modification of antisense oligonucleotides may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methyhribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides.

In some embodiments, an oligonucleotide sugar moiety is a modified sugar moiety. In some embodiments, the modified sugar moiety can be a sugar moiety which is a conformationally-strained sugar. In some embodiments, the conformationally-strained sugar can be a locked nucleotide (locked nucleic acid, or LNA). In some embodiments, the locked nucleotide can be selected from one of the following types: 2′-O—CH₂-4′ (oxy-LNA), 2′-CH₂—CH₂-4′ (methylene-LNA), 2′-NH—CH₂-4′(amino-LNA), 2′-N(CH₃)— CH₂-4′(methylamino-LNA), 2′-S—CH₂-4′ (thio-LNA), and 2′-Se—CH₂-4′ (seleno-LNA). In some embodiments, the conformationally-strained sugar can be a bridged nucleic acid (BNA). Some conformationally-strained sugar can be a locked nucleic acid are shown in Formula III and Formula IV in U.S. Pat. No. 10,465,188, herein incorporated by reference.

Synthesis of antisense oligonucleotides can be performed. See e.g. Stein C. A. and Krieg A. M. (eds), Applied Antisense Oligonucleotide Technology, 1998 (Wiley-Liss).

### Hybridization

Hybridization occurs between a gapmer compound and a target nucleic acid (SEQ ID NO. 1). The most common mechanism of hybridization involves hydrogen bonding (e.g., Watson-Crick, Hoogsteen or reversed Hoogsteen hydrogen bonding) between complementary nucleobases of the nucleic acid molecules. Hybridization can occur under varying conditions. Stringent conditions are sequence-dependent and are determined by the nature and composition of the nucleic acid molecules to be hybridized.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. Nucleosides comprise chemically modified ribofuranose ring moieties. Examples of chemically modified ribofuranose rings include, without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (WO2008/101157 for other disclosed 5′,2′-bis substituted nucleosides) or replacement of the ribosyl ring oxygen atom with S with further substitution at the 2′-position (U.S. Patent Application 2005/0130923) or alternatively 5′-substitution of a BNA (WO2007/134181 wherein LNA is substituted with for example a 5′-methyl or a 5′-vinyl group).

Antisense oligonucleotides may be part of compositions which may comprise oligonucleotides to more than one region of IL-1β eRNA (SEQ ID NO: 1).

When specific proteins are referred to herein, derivatives, variants, and fragments are contemplated and included. Protein derivatives and variants are well understood to those of skill in the art and can involve insertional, substitutional or deletional amino acid sequence variants known in the art.

### Modified Nucleotide Bases

Modified nucleotide bases include Formula Ia Formula Ib, Formula IIa, or Formula IIb:

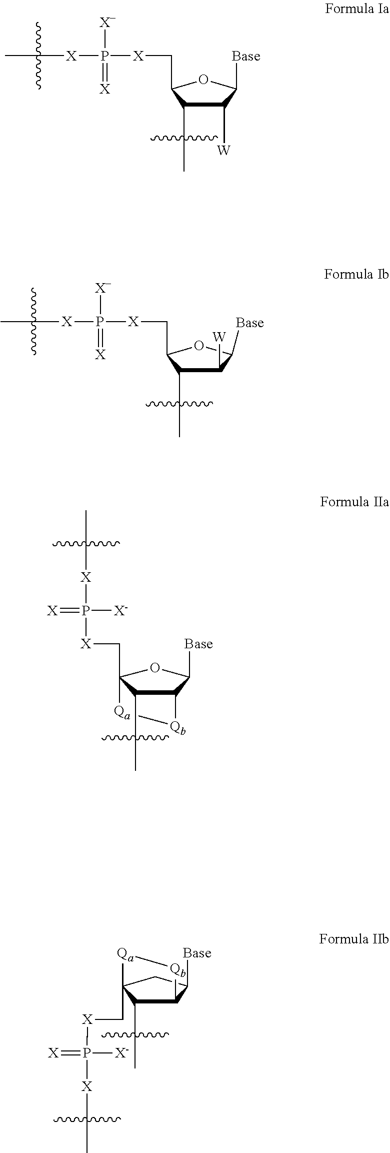

wherein
each X is independently O or S, wherein 0, 1, or 2 instances of X is S;
each W is independently H, OH, halo, or —O—C₁₋₆ alkyl, wherein the alkyl is optionally substituted with up to three instances of C₁₋₄ alkyl, C₁₋₄ alkoxy, halo, amino, CN, NO₂, or OH; each Qₐ is independently a bifunctional C₁₋₆ alkylene, optionally substituted with up to two instances of C₁₋₄ alkyl, C₁₋₄ alkoxy, halo, or OH; and each Q_{b} is independently a bond or a bifunctional moiety selected from —O—, —S—, —N—O—, —N(R)—, —C(O)—, —C(O)O—, and —C(O)N(R)—, wherein R is an unsubstituted C₁₋₄ alkyl.

In one embodiment, each X is O. In another embodiment, one instance of X is S.

In one embodiment, the gapmer comprises one or more nucleotides of Formula Ia or Formula Ib, wherein W is halo. In a further embodiment, W is fluoro. In another further embodiment, the gapmer comprises one or more nucleotides of Formula Ia. In another further embodiment, the gapmer comprises one or more nucleotides of Formula Ib.

In one embodiment, the gapmer comprises one or more nucleotides of Formula Ia or Formula Ib, wherein W is —O—C₁₋₆ alkyl, wherein the alkyl is optionally substituted with up to three instances of C₁₋₄ alkyl, C₁₋₄ alkoxy, halo, amino, or OH. In a further embodiment, W is —O—C₁₋₆ alkyl, wherein the alkyl is optionally substituted with C₁₋₄ alkoxy. In a further embodiment, W is an unsubstituted —O—C₁₋₆ alkyl. In another further embodiment, W is —O—C₁₋₆ alkyl, wherein the alkyl is substituted with C₁₋₄ alkoxy. In a further embodiment, W is selected from methoxy and —O—CH₂CH₂—OCH₃. In one embodiment, the gapmer comprises one or more nucleotides of Formula Ia. In another embodiment, the gapmer comprises one or more nucleotides of Formula Ib.

In one embodiment, the gapmer comprises one or more β-D nucleotides of Formula IIa or α-L nucleotides of Formula IIb, wherein Qₐ is an unsubstituted bifunctional C₁₋₆ alkylene, and Q_{b} is a bond or a bifunctional moiety selected from —O—, —S—, —N—O—, and —N(R)—. In a further embodiment, Qₐ is selected from —CH₂—, —CH₂—CH₂—, —CH(CH₃)—, —CH₂—CH₂(CH₃)—, and Q_{b} is a bond or a bifunctional moiety selected from —O—, —S—, —N(R)—O—, and —N(R)—, wherein R is H or C₁₋₆ alkyl.

In one embodiment of Formula IIa or Formula IIb, Qₐ is —CH₂— and Q_{b} is —O—. In another embodiment of Formula IIa or Formula IIb, Qₐ is —CH₂—CH₂— and Q_{b} is —O—. In another embodiment of Formula IIa or Formula IIb, Qₐ is —CH₂— and Q_{b} is —N(R)—O—, wherein R is H or C₁₋₆ alkyl. In another embodiment of Formula IIa or Formula IIb, Qₐ is —CH(CH₃)— and Q_{b} is —O—. In another embodiment of Formula IIa or Formula IIb, Qₐ is —CH₂— and Q_{b} is —S—. In another embodiment of Formula IIa or Formula IIb, Qₐ is —CH₂— and Q_{b} is —N(R)—, wherein R is H or C₁₋₆ alkyl. In another embodiment of Formula IIa or Formula IIb, Qₐ is —CH₂—CH(CH₃)— and Q_{b} is a bond.

In some embodiments, the gapmer comprises one or more nucleotides selected from the following modified nucleotides:

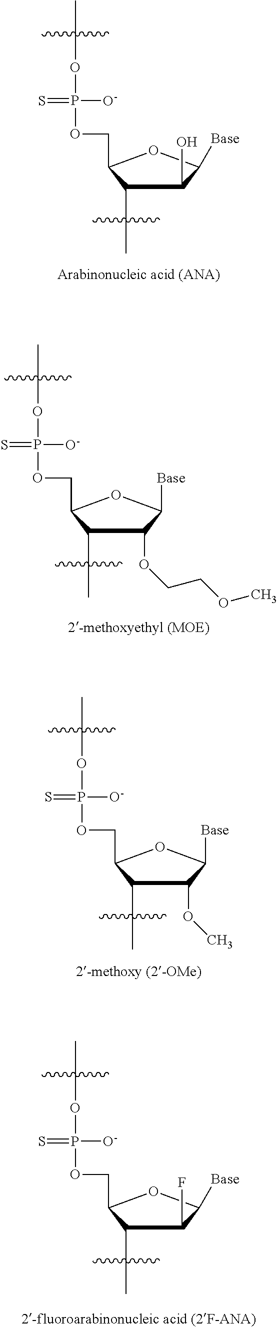

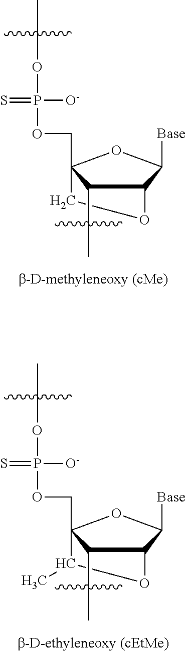

Many other bicyclo and tricyclo sugar surrogate ring systems can be used to modify nucleosides for incorporation into antisense compounds (see, for example, review article: Leumann, Boorg. Med. Chem., 2002, 10, 841-854).

### Administration

The gapmers described herein may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal, oral or parenteral. The compounds and compositions described herein can be delivered in a manner to target a particular tissue, such as the bone marrow or brain. The compounds and compositions described herein are administered parenterally. “Parenteral administration” means administration through injection or infusion. Parenteral administration includes subcutaneous administration, intravenous administration, intramuscular administration, intraarterial administration, intraperitoneal administration, or intracranial administration, e.g. intracerebral administration, intrathecal administration, intraventricular administration, ventricular administration, intracerebroventricular administration, cerebral intraventricular administration or cerebral ventricular administration. Administration can be continuous, or chronic, or short or intermittent.

Parenteral administration is also by infusion. Infusion can be chronic or continuous or short or intermittent, with a pump or by injection. In some embodiments, parenteral administration is subcutaneous.

### Formulations

The pharmaceutical compositions of this invention include, for example, compositions comprising an IL-1β eRNA transcriptional inhibitor gapmer compound. The pharmaceutical formulations of this invention may further comprise one or more pharmaceutically acceptable excipients.

The pharmaceutical formulations of this invention may further comprise one or more pharmaceutically acceptable excipients.

The IL-1β eRNA transcriptional inhibitor gapmer compounds may be present in the formulation in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 80%, 85%, or 90%, e.g. at least about 88%, at least about 90, 95 or 98%, or at least about 99% of a oligonucleotide, or dry mass of the preparation.

Pharmaceutically acceptable diluents, carriers and/or excipients include those suitable for veterinary use as well as human pharmaceutical use. By way of example, diluents, carriers and/or excipients include solutions, solvents, dispersion media, delay agents, polymeric and lipidic agents, emulsions and the like. By way of further example, suitable liquid carriers, especially for injectable solutions, include water, aqueous saline solution, aqueous dextrose solution, and the like, and vehicles such as liposomes being also especially suitable for administration of agents.

Suitable carriers and diluents include buffered, aqueous solutions, saline, dextrose, glycerol, isotonic saline solutions, for example phosphate-buffered saline, isotonic water, and the like and combinations thereof. In some embodiments, carriers may include propylene glycol, dimethyl isosorbide, and water, and even more particularly, phosphate buffered saline, isotonic water, deionized water, monofunctional alcohols and symmetrical alcohols. In some embodiments pharmaceutically acceptable carrier or diluent may be or contain a thermosetting poloxamer (which may be a liquid or gel, depending on the temperature), a carboxycellulose (e.g. carboxymethylcellulose), a collagen (e.g., a Type I collagen), a collagenous material comprising tropocollagen, a hyaluronan or derived-hyaluronic acid, and/or an oil (e.g., Emu oil). Suitable carriers can be large, slowly metabolized macromolecules such as proteins, polysaccharides, polylactic acids, polyglycolic acids, polymeric amino acids, and amino acid copolymers.

Compositions may take the form of any standard known dosage form including tablets, pills, capsules, semisolids, powders, sustained release formulation, solutions, suspensions, elixirs, aerosols, liquids for injection, gels, creams, transdermal delivery devices (for example, a transdermal patch), inserts such as ocular inserts, or any other appropriate compositions.

Preferably the IL-1β eRNA transcriptional inhibitor gapmer compound is combined with a pharmaceutically acceptable carrier or diluent to produce a pharmaceutical composition.

Pharmaceutically acceptable salts can also be present, e.g., mineral acid salts such as hydrochlorides, hydrobromides, phosphates, sulfates, and the like; and the salts of organic acids such as citrates, acetates, propionates, malonates, benzoates, and the like.

In addition, if desired substances such as wetting or emulsifying agents, stabilizing or pH buffering agents, or preservatives may also be present. In some embodiments, the pharmaceutical compositions of this invention will comprise suitable acceptable buffers, such as acetate buffers, citrate buffers, phosphate buffers, borate buffers and mixtures thereof. In some embodiments, the buffers useful in the present invention include boric acid, sodium borate, sodium phosphates, including mono, di- and tri-basic phosphates, such as sodium phosphate monobasic monohydrate and sodium phosphate dibasic heptahydrate, and mixtures thereof. In some embodiments, the preservative may be stabilized chlorine dioxide, cationic polymers or quaternary ammonium compounds. In some embodiments the pharmaceutical compositions may also comprise wetting agents, nutrients, viscosity builders, antioxidants, and the like, for example, disodium ethylene diamine tetraacetate, alkali metal hexametaphosphate, citric acid, sodium citrate, sodium metabisulfite, sodium thiosulfate, N-acetylcysteine, butylated hydroxyanisole, butylated hydroxytoluene, polyvinyl alcohol, polyoxamers, polyvinyl pyrrollidone, hydroxypropyl methyl cellulose, hydroxyethylmethyl cellulose, and mixtures thereof and mixtures thereof. In some embodiments, the pharmaceutical formulations of this invention will not include a preservative. In some embodiments, the IL-1β eRNA transcriptional inhibitor gapmer composition or formulation comprises sodium phosphate dibasic heptahydrate or potassium phosphate, monobasic or both.

Uptake of nucleic acids by mammalian cells may be enhanced by the of known transfection techniques including the use of transfection agents. Such techniques may be used with certain IL-1β eRNA transcriptional inhibitor gapmer compounds. Examples of useful transfection agents include cationic agents (for example calcium phosphate and DEAE-dextran) and lipofectants (for example Lipofectam™ and Transfectam™), and surfactants.

The compositions may be formulated in accordance with standard techniques known in the art, including those as may be found in such standard references as Gennaro A R: Remington: The Science and Practice of Pharmacy, 20^{th} ed., Lippincott, Williams & Wilkins, 2000, for example.

Any container suitable for storing and/or administering a pharmaceutical composition may be used in a combination product of the invention. Suitable containers will be appreciated by persons skilled in the art. By way of example, such containers include vials and syringes. The containers may be suitably sterilized and hermetically sealed.

Such compositions comprise a pharmaceutically acceptable solvent, such as water or saline, diluent, carrier, or adjuvant. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial (intrathecal or intraventricular, administration).

The compounds may also be admixed, conjugated or otherwise associated with other molecules, molecule structures or mixtures of compounds, as for example, liposomes, receptor-targeted molecules, or other formulations, for assisting in uptake, distribution and/or absorption.

The term “pharmaceutically acceptable carriers” refers to physiologically and pharmaceutically acceptable carriers of the compounds i.e., carriers that retain the desired biological activity of the parent compound and do not impart undesired toxicological effects thereto. For oligonucleotides, preferred examples of pharmaceutically acceptable carriers and their uses are further described in U.S. Pat. No. 6,287,860, which is incorporated by reference herein. Sodium carriers have been shown to be suitable forms of oligonucleotide drugs.

Formulations include liposomal formulations. The term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes which refers to liposomes comprising one or more specialized lipids that, when incorporated into liposomes, result in enhanced circulation lifetimes relative to liposomes lacking such specialized lipids. Liposomes and their uses are further described in U.S. Pat. No. 6,287,860, which is incorporated by reference herein.

Preferred formulations for topical administration include those in which the oligonucleotides are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

### Lipid Nanoparticles

LNPs are multi-component systems that typically consist of an ionizable amino lipid, a phospholipid, cholesterol, and a polyethylene glycol (PEG)-lipid, with all of the components contributing to efficient delivery of the nucleic acid drug cargo and stability of the particle (Schroeder et al., J. Intern. Med. 2010; 267:9-21). The cationic lipid electrostatically condenses the negatively charged RNA into nanoparticles and the use of ionizable lipids that are positively charged at acidic pH is thought to enhance endosomal escape. Formulations for delivery, both clinically and non-clinically, are predominantly based on cationic lipids such as DLin-MC3-DMA (MC3). (Kanasty et al. Nat. Mater. 2013; 12:967-977; and Xue et al. Curr. Pharm. Des. 2015; 21:3140-3147).

Further LNP's include a nanoemulsion having a perfluorcarbon component (a) consisting of at least one least one perfluorcarbon compound, an emulsifying component (b) such as phospholipids and optionally helper lipids, and an endocytosis enhancing component (c) that comprises at least one compound inducing cellular uptake of the nanoemulsion. A perfluorcarbon compound of component (a) is preferably selected from compounds having the structure CₘF₂ₘ₊₁X, XCₘF₂ₘX, XCₙF₂ₙOCₒF₂ₒX, N(CₒF₂ₒX)₃ and N(CₒF₂ₒ+1)₃, wherein m is an integer from 3 to 10, n and o are integers from 1 to 5, and X is independently from further occurrence selected from Cl, Br and I. Examples of perfluorcarbon compounds are perfluorooctyl bromide and perfluorotributylamine.

Examples of the emulsifying agents include phospholipids, such as the phospholipid compound represented by the formula I:

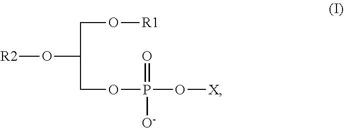

wherein, R¹ and R² are independently selected from H and C₁₆₋₂₄ acyl residues, which may be saturated or unsaturated and may carry 1 to 3 residues R³ and wherein one or more of the C-atoms may be substituted by O or NR⁴, and X is selected from H, —(CH₂)ₚ—N(R⁴)₃⁺, —(CH₂)ₚ—CH(N(R⁴)₃⁺)—COO⁻, —(CH₂)ₚ—CH(OH)—CH₂OH and —CH₂(CHOH)ₚ—CH₂OH (wherein p is an integer from 1 to 5); R³ is independently selected from H, lower alkyl, F, Cl, CN und OH; and R⁴ is independently selected from H, CH₃ und CH₂CH₃, or a pharmacologically acceptable carrier thereof.

Following subcutaneous (s.c.) administration, LNPs and their mRNA cargo are expected to be largely retained at the site of injection, resulting in high local concentrations. Since LNPs are known to be pro-inflammatory, largely attributed to the ionizable lipid present in the LNPs, (Sabnis et al. Mol. Ther. 2018; 26:1509-1519) then it would be expected that s.c. administration of mRNA formulated in LNPs would be associated with dose-limiting inflammatory responses. Co-administration of dexamethasone with LNP reduces the immune-inflammatory response following i.v. administration (Abrams et al. Mol. Ther. 2010; 18:171-180). And Chen et al. (J. Control. Release. 2018; 286:46-54.) showed reduced immune stimulation following systemic administration by incorporating lipophilic dexamethasone prodrugs within LNP-containing nucleic acids.

### Dosing

Optimal dosing schedules are calculated from measurements of drug accumulation in the body of the patient. Optimum dosages vary depending on the relative potency of individual oligonucleotides, and can generally be estimated based on EC50s found to be effective in in vitro and in vivo animal models. In general, dosage is from 0.01 μg to 100 g per kg of body weight, and may be given once or more daily, weekly, monthly or yearly, or at desired intervals. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily.

Therapeutically effective amounts include but are not limited to the doses described herein. Described doses and other therapeutically effective amounts are administered in one or more of the therapeutically effective dose regimens described herein.

### Articles of Manufacture/Kits

In another embodiment of the invention, an article of manufacture, or “kit”, containing materials useful for inhibiting IL-1β eRNA is provided. The kit comprises a container with a composition comprising one or more modulators, e.g. an IL-1β0 eRNA modulator such as a IL-1β eRNA transcriptional inhibitor gapmer compound as described herein, for example. Suitable containers include, e.g., bottles, vials, etc. The container may be formed from a variety of materials such as glass or plastic. The kit may also comprise a pharmaceutically acceptable carrier. In some embodiments the kit may also include components for administering the compositions, for example, a syringe, needle, microneedle, etc.

### Manufacture

The oligonucleotides of this invention can be manufactured using solid-phase chemistries for synthesizing oligonucleotides, chemistries known in the art for synthesizing and preparing peptides and peptidomimetics, and chemistries know in the art for synthesizing organic compounds. In one aspect, the formulations of this invention will comprise a salt of the oligonucleotides of this invention, such as the sodium salt of the oligonucleotides of this invention. The kit may also comprise a pharmaceutically acceptable carrier.

In some embodiments, the formulations of this invention are substantially pure. By substantially pure is meant that the formulations comprise less than about 10%, 5%, or 1%, and preferably less than about 0.1%, of any nucleotide or non-nucleotide impurity. In some embodiments the total impurities, including metabolites of the transcriptional inhibitor gapmer type of ASO (antisense oligonucleotide) compound, will be not more than 15%. In some embodiments the total impurities, including metabolites of the IL-1B eRNA transcriptional inhibitor gapmer type of ASO (antisense oligonucleotide) compound, will be not more than 12%. In some embodiments the total impurities, including metabolites of the IL-1B eRNA transcriptional inhibitor gapmer type of ASO (antisense oligonucleotide) compound, will be not more than 11%. In other embodiments the total impurities, including metabolites of the IL-1B eRNA transcriptional inhibitor gapmer type of ASO (antisense oligonucleotide) compound, will be not more than 10%.

Sterile compositions comprising the IL-1B eRNA transcriptional inhibitor gapmer type of ASO (antisense oligonucleotide) compounds of this invention prepared using aseptic processing by dissolving the IL-1B eRNA transcriptional inhibitor gapmer type of ASO (antisense oligonucleotide) compound in the formulation vehicle. In one embodiment, the formulation may also be sterilized by filtration. Excipients used in the manufacture of the formulations of this invention are widely used in pharmaceutical products and released to pharmacopeial standards.

### EXAMPLES

### Example 1

This example provides a screening system for in vitro assays of candidate gapmers for inhibiting gene transcription regulated by IL-1β eRNA. The effect of candidate gapmer compounds were screened for target nucleic acid expression (e.g., messenger RNA) by real-time polymerase chain reaction (RT-PCR).

THP-1 human monocytic cell line (derived from an acute leukemia patient) was obtained from InvivoGen. THP1 cells were maintained in complete media, which is composed of RPMI 1640, 1% (2 mM) GlutaMAX L-glutamine supplement, 25 mM HEPES, 10% FBS, 100 μg/ml Normocin, Pen-Strep (100 U/ml), Blasticidin (10 μg/ml) and Zeocin (100 μg/ml). Prior to seeding for the screen, the THP-1 monocyte culture was split by 50% to enable the cells to re-enter an exponential growth phase. 250 000 cells were seeded per well in quadruplicate in 96-well plates with 180 μL of complete medium in each well. Each gapmer compound tested was added to the THP-1 cells at a final concentration of 10 μM and mixed gently. Plates were incubated at 37° C. at 5% CO2 for 24 hours. Then, LPS (10 ng/mL) was added to each well, and plates were incubated at 37° C. at 5% CO2 for another 24 hours.

Antisense modulation of IL-1β eRNA expression on specified genes was assayed by real-time PCR (RT-PCR). RNA analysis was performed on total cellular RNA or poly(A)+mRNA. RNA was isolated and prepared using TRIZOL® Reagent (Thermo Fisher Scientific) and Direct-zol RNA Miniprep Kit (Zymo Research) according to the manufacturer's recommended protocols.

Quantitation of target RNA levels was accomplished by quantitative real-time PCR using a CFX Real-time qPCR detection system (Bio Rad). Prior to real-time PCR, the isolated RNA was subjected to a reverse transcriptase (RT) reaction, which produces complementary DNA (cDNA) that is then used as the substrate for the real-time PCR amplification. RT reaction reagents and real-time PCR reagents were obtained from Thermo Fisher Scientific, and protocols for their use are provided by the manufacturer. Gene (or RNA) target quantities obtained by real time PCR were normalized using expression levels of stably expressed housekeeping genes such as HPRT or RPL37A. Total RNA was quantified using a Qubit Fluorometer (Invitrogen/Scientific) and a Qubit RNA HS Assay Kit (Thermo Fisher Scientific Cat. No. Q32852) in accordance with the manufacturer's protocol. The Qubit Flourometer was calibrated with standards.

A series of gapmer compounds are shown in Table 1. These gapmers were designed to target different regions of the human IL-1β eRNA (SEQ ID NO. 1). The gapmer compounds in Table 1 are chimeric oligonucleotides (“gapmers”) having different configurations. For instance, gapmers with a configuration of 20 (5-10-5) nucleotides in length were composed of a central “gap” region of ten 2′-deoxynucleotides, which was flanked on both sides (5′ and 3′ directions) by five-nucleotide “wings”. These wings were composed of 2′-methoxyethyl (2′-MOE) sugar modified nucleosides. The internucleotide (backbone) linkages were phosphorothioate throughout the entire oligonucleotide sequence. Cytidine residues were 5-methylcytidines unless indicated otherwise, in which case they were cytidines residues. Gapmers with a configuration of 16 (3-10-3) nucleotides in length were composed of a central “gap” region comprising ten 2′-deoxynucleotides, which was flanked on both sides (5′ and 3′ directions) by three-nucleotide “wings”. In some embodiments, the wings were composed of locked nucleic acid (LNA) modified nucleosides employing the cMe locked nucleic acid modification. The internucleotide (backbone) linkages were phosphorothioate throughout the entire oligonucleotide sequence. Cytidine residues were 5-methylcytidines unless indicated otherwise, in which case they were cytidine residues.

Gapmer compounds used are shown in Table 1. Abbreviations of the nucleoside modification in Table 1: M=2′-methoxyethyl (2′-MOE) modified nucleoside; L=locked nucleic acid (LNA) modified nucleoside (cMe); d=2′-deoxynucleosides.

TABLE 1

| | | | | Complementary |
|---|---|---|---|---|
| | | | | position to |
| | | | Sequence | human IL-1β |
| SEQ | Con- | Nucleoside | of gapmer | eRNA |
| ID NO. | figuration | Modification | compound | (SEQ ID NO. 1) |
| 3 | 5-10-5 | MMMMMddddddddddMM | CACCTCTCCTGCAT | 736-755 |
| | | MMM | ATGACG | |
| 4 | 5-10-5 | MMMMMddddddddddMM | CCTCTCCTGCATAT | 734-753 |
| | | MMM | GACGTC | |
| 5 | 5-10-5 | MMMMMddddddddddMM | CTTCCATCCCAGGG | 210-229 |
| | | MMM | AAAGGT | |
| 6 | 5-10-5 | MMMMMddddddddddMM | GCTTTTCGGACTGG | 780-799 |
| | | MMM | TCCTTC | |
| 7 | 5-10-5 | MMMMMddddddddddMM | CTGCTGCTGCTACT | 37-56 |
| | | MMM | GCTACT | |
| 8 | 5-10-5 | MMMMMddddddddddMM | TTGCATCTTCCATC | 216-235 |
| | | MMM | CCAGGG | |
| 9 | 5-10-5 | MMMMMddddddddddMM | TCTGGTTCTGCTGC | 44-63 |
| | | MMM | TGCTAC | |
| 10 | 5-10-5 | MMMMMddddddddddMM | CCACCTCTCCTGCA | 737-756 |
| | | MMM | TATGAC | |
| 11 | 5-10-5 | MMMMMMMMdddddddddd | GATTGCAGCTCAC | 997-1016 |
| | | MMMMM | AGGGGTGGATT | |
| 12 | 5-10-5 | MMMMMMMMdddddddddd | CTCAGCAATGTAA | 870-889 |
| | | MMMMM | TGTCCCCCTC | |
| 13 | 5-10-5 | MMMMMMMMdddddddddd | CACAAATCTGCCG | 1087-1106 |
| | | MMMMM | CAATGCCCAC | |
| 14 | 5-10-5 | MMMMMMMMdddddddddd | TGCGGTTCTGCTGC | 41-60 |
| | | MMMMM | TGCTACTGC | |
| 15 | 5-10-5 | MMMMMMMMdddddddddd | CTTTCTTGGCCAAT | 1342-1361 |
| | | MMMMM | TCCCTTCTT | |
| 16 | 5-10-5 | MMMMMMMMdddddddddd | CGTACCTCTCCTGC | 735-754 |
| | | MMMMM | ATATGACGT | |
| 17 | 5-10-5 | MMMMMMMMdddddddddd | ATGAGAGACTGAG | 89-108 |
| | | MMMMM | TTGGCAAATGA | |
| 18 | 5-10-5 | MMMMMMMMdddddddddd | AGGACAGACGAGT | 487-506 |
| | | MMMMM | TACTCCCAGG | |
| 19 | 5-10-5 | MMMMMMMMdddddddddd | GCTCTGCTGCTACT | 34-53 |
| | | MMMMM | GCTACTGCT | |
| 20 | 5-10-5 | MMMMMMMMdddddddddd | GAACATCAACCCC | 455-474 |
| | | MMMMM | CTCCACAGAA | |
| 21 | 5-10-5 | MMMMMMMMdddddddddd | AGGTCTTCCATCCC | 211-230 |
| | | MMMMM | AGGGAAAGG | |
| 22 | 5-10-5 | MMMMMMMMdddddddddd | CTGTGCTGCTGCTA | 36-55 |
| | | MMMMM | CTGCTACTG | |
| 23 | 5-10-5 | MMMMMMMMdddddddddd | ATCTGGCCAATTCC | 1339-1358 |
| | | MMMMM | CTTCTTATC | |
| 24 | 5-10-5 | MMMMMddddddddddMM | TTCATCTTGGCCA | 1346-1365 |
| | | MMM | ATTCCCT | |
| 25 | 5-10-5 | MMMMMddddddddddMM | TGGATCCATCTCT | 1271-1290 |
| | | MMM | TAAGAAA | |
| 26 | 5-10-5 | MMMMMddddddddddMM | ATCTTGGCCAATT | 1343-1362 |
| | | MMM | CCCTTCT | |
| 27 | 5-10-5 | MMMMMddddddddddMM | CTATTCCCTCCCTC | 1786-1805 |
| | | MMM | TTAAGA | |
| 28 | 5-10-5 | MMMMMddddddddddMM | CCCGCAAAAAAA | 658-677 |
| | | MMM | AAGGCCTC | |
| 29 | 5-10-5 | MMMMMddddddddddMM | TTCTGGTTCTGCT | 45-64 |
| | | MMM | GCTGCTA | |
| 30 | 5-10-5 | MMMMMddddddddddMM | GGGGAAATTCTGG | 52-71 |
| | | MMM | TTCTGCT | |
| 31 | 5-10-5 | MMMMMddddddddddMM | CGGGGAAATTCTG | 53-72 |
| | | MMM | GTTCTGC | |
| 32 | 5-10-5 | MMMMMddddddddddMM | GCAAATGAGAGCC | 77-96 |
| | | MMM | TGA GACA | |
| 33 | 5-10-5 | MMMMMddddddddddMM | ACTGAGTTGGCAA | 86-105 |
| | | MMM | ATGAGAG | |
| 34 | 5-10-5 | MMMMMddddddddddMM | TGCCAATAAAAAT | 107-126 |
| | | MMM | ACTTAGA | |
| 35 | 5-10-5 | MMMMMddddddddddMM | CTGCCAATAAAAA | 108-127 |
| | | MMM | TACTTAG | |
| 36 | 5-10-5 | MMMMMddddddddddMM | ATAGCTATTTTAT | 126-145 |
| | | MMM | TTTTCCT | |
| 37 | 5-10-5 | MMMMMddddddddddMM | ATTTCACTCATAG | 135-154 |
| | | MMM | CTATTTT | |
| 38 | 5-10-5 | MMMMMddddddddddMM | CTAATGAATTATT | 145-164 |
| | | MMM | TCACTCA | |
| 39 | 5-10-5 | MMMMMddddddddddMM | TCTAATGAATTAT | 146-165 |
| | | MMM | TTCACTC | |
| 40 | 5-10-5 | MMMMMddddddddddMM | TAAACACAAAATT | 173-192 |
| | | MMM | GATGGAG | |
| 41 | 5-10-5 | MMMMMddddddddddMM | TTAAACACAAAAT | 174-193 |
| | | MMM | TGATGGA | |
| 42 | 5-10-5 | MMMMMddddddddddMM | TTTAAACACAAAA | 175-194 |
| | | MMM | TTGATGG | |
| 43 | 5-10-5 | MMMMMddddddddddMM | CCCAGGGAAAGGT | 203-222 |
| | | MMM | AAAGAGA | |
| 44 | 5-10-5 | MMMMMddddddddddMM | TTTGCATCTTCCAT | 217-236 |
| | | MMM | CCCAGG | |
| 45 | 5-10-5 | MMMMMddddddddddMM | CAAGCATACAATA | 271-290 |
| | | MMM | TACCCAC | |
| 46 | 5-10-5 | MMMMMddddddddddMM | ATGGCTGGAACTC | 283-302 |
| | | MMM | AAGCATA | |
| 47 | 5-10-5 | MMMMMddddddddddMM | AATGGCTGGAACT | 284-303 |
| | | MMM | CAAGCAT | |
| 48 | 5-10-5 | MMMMMddddddddddMM | CGAGGCTCAAGGA | 888-907 |
| | | MMM | GAACAAG | |
| 49 | 5-10-5 | MMMMMddddddddddMM | CCGCAATGCCCAC | 1080-1099 |
| | | MMM | ACAGTCA | |
| 50 | 5-10-5 | MMMMMddddddddddMM | ATAATACAGCGAC | 1153-1172 |
| | | MMM | GACAATAATAA | |
| 51 | 5-10-5 | MMMMMddddddddddMM | TCTGTTCTTCTGGA | 1278-1297 |
| | | MMM | TCCATCTCT | |
| 52 | 5-10-5 | MMMMMddddddddddMM | CTTTCCACCTCCCT | 1663-1682 |
| | | MMM | TCCTTGCTT | |
| 53 | 5-10-5 | MMMMMddddddddddMM | GAAAAAAGTGGAG | 1758-1777 |
| | | MMM | GCTCAGAGAAA | |
| 54 | 3-10-3 | LLLddddddddddLLL | TCTACACACATGG | 325-340 |
| | | | CAG | |
| 55 | 3-10-3 | LLLddddddddddLLL | GTCTACACACATG | 326-341 |
| | | | GCA | |
| 56 | 3-10-3 | LLLddddddddddLLL | AGTCTACACACAT | 327-342 |
| | | | GGC | |
| 57 | 3-10-3 | LLLddddddddddLLL | AAGTCTACACACA | 328-343 |
| | | | TGG | |
| 58 | 3-10-3 | LLLddddddddddLLL | TCTATAGAAAGTC | 336-351 |
| | | | TAC | |
| 59 | 3-10-3 | LLLddddddddddLLL | GTCTATAGAAAGT | 337-352 |
| | | | CTA | |
| 60 | 3-10-3 | LLLddddddddddLLL | AGTCTATAGAAAG | 338-353 |
| | | | TCT | |
| 61 | 3-10-3 | LLLddddddddddLLL | ACAGTCTATAGAA | 340-355 |
| | | | AGT | |
| 62 | 3-10-3 | LLLddddddddddLLL | CACAGTCTATAGA | 341-356 |
| | | | AAG | |
| 63 | 3-10-3 | LLLddddddddddLLL | AAGTGGCAGGTCG | 363-378 |
| | | | GGT | |
| 64 | 3-10-3 | LLLddddddddddLLL | GCGGGGAAATTCT | 58-73 |
| | | | GGT | |
| 65 | 3-10-3 | LLLddddddddddLLL | TGCGGGGAAATTC | 59-74 |
| | | | TGG | |
| 66 | 3-10-3 | LLLddddddddddLLL | GTGCGGGGAAATT | 60-75 |
| | | | CTG | |
| 67 | 3-10-3 | LLLddddddddddLLL | CGTGCGGGGAAAT | 61-76 |
| | | | TCT | |
| 68 | 3-10-3 | LLLddddddddddLLL | ACACGTGCGGGGA | 64-79 |
| | | | AAT | |
| 69 | 3-10-3 | LLLddddddddddLLL | GACACGTGCGGGG | 65-80 |
| | | | AAA | |
| 70 | 3-10-3 | LLLddddddddddLLL | TCGGGTTTAGCAC | 353-368 |
| | | | ACA | |
| 71 | 3-10-3 | LLLddddddddddLLL | GGTCGGGTTTAGC | 355-370 |
| | | | ACA | |
| 72 | 3-10-3 | LLLddddddddddLLL | AGGTCGGGTTTAG | 356-371 |
| | | | CAC | |
| 73 | 3-10-3 | LLLddddddddddLLL | GCAGGTCGGGTTT | 358-373 |
| | | | AGC | |
| 74 | 3-10-3 | LLLddddddddddLLL | GGCAGGTCGGGTT | 359-374 |
| | | | TAG | |
| 75 | 3-10-3 | LLLddddddddddLLL | TGGCAGGTCGGGT | 360-375 |
| | | | TTA | |
| 76 | 3-10-3 | LLLddddddddddLLL | CAAATTGGGGTAG | 416-431 |
| | | | GGC | |
| 77 | 3-10-3 | LLLddddddddddLLL | GCCGTCCTTTAAC | 472-487 |
| | | | CAT | |
| 78 | 3-10-3 | LLLddddddddddLLL | TACTCCCAGGCCG | 481-496 |
| | | | TCC | |
| 79 | 3-10-3 | LLLddddddddddLLL | TTACTCCCAGGCC | 482-497 |
| | | | GTC | |
| 80 | 3-10-3 | LLLddddddddddLLL | CGAGTTACTCCCA | 486-501 |
| | | | GGC | |
| 81 | 3-10-3 | LLLddddddddddLLL | ACGAGTTACTCCC | 487-502 |
| | | | AGG | |
| 82 | 3-10-3 | LLLddddddddddLLL | GACGAGTTACTCC | 488-503 |
| | | | CAG | |
| 83 | 3-10-3 | LLLddddddddddLLL | AGACGAGTTACTC | 489-504 |
| | | | CCA | |
| 84 | 3-10-3 | LLLddddddddddLLL | CAGACGAGTTACT | 490-505 |
| | | | CCC | |
| 85 | 3-10-3 | LLLddddddddddLLL | ACAGACGAGTTAC | 491-506 |
| | | | TCC | |
| 86 | 3-10-3 | LLLddddddddddLLL | CCCTAGTACAGAC | 498-513 |
| | | | GAG | |
| 87 | 3-10-3 | LLLddddddddddLLL | GCCCTAGTACAGA | 499-514 |
| | | | CGA | |
| 88 | 3-10-3 | LLLddddddddddLLL | GGCCCTAGTACAG | 500-515 |
| | | | ACG | |
| 89 | 3-10-3 | LLLddddddddddLLL | GGGCCCTAGTACA | 501-516 |
| | | | GAC | |
| 90 | 3-10-3 | LLLddddddddddLLL | TGGGCCCTAGTAC | 502-517 |
| | | | AGA | |
| 91 | 3-10-3 | LLLddddddddddLLL | CTGGGCCCTAGTA | 503-518 |
| | | | CAG | |
| 92 | 3-10-3 | LLLddddddddddLLL | CCTGGGCCCTAGT | 504-519 |
| | | | ACA | |
| 93 | 3-10-3 | LLLddddddddddLLL | TCCTGGGCCCTAG | 505-520 |
| | | | TAC | |
| 94 | 3-10-3 | LLLddddddddddLLL | GTAGAAAGGCCCT | 589-604 |
| | | | AAC | |
| 95 | 3-10-3 | LLLddddddddddLLL | TCGGCTTGCTCCC | 680-695 |
| | | | CCA | |
| 96 | 3-10-3 | LLLddddddddddLLL | GTCGGCTTGCTCC | 681-696 |
| | | | CCC | |
| 97 | 3-10-3 | LLLddddddddddLLL | TTAGTCGGCTTGC | 684-699 |
| | | | TCC | |
| 98 | 3-10-3 | LLLddddddddddLLL | ATTAGTCGGCTTG | 685-700 |
| | | | CTC | |
| 99 | 3-10-3 | LLLddddddddddLLL | GACTCACTACACC | 752-767 |
| | | | ACC | |
| 100 | 3-10-3 | LLLddddddddddLLL | AATCCCGTTGTGA | 763-778 |
| | | | CTC | |
| 101 | 3-10-3 | LLLddddddddddLLL | CTAATCCCGTTGT | 765-780 |
| | | | GAC | |
| 102 | 3-10-3 | LLLddddddddddLLL | CCTTCTAATCCCG | 769-784 |
| | | | TTG | |
| 103 | 3-10-3 | LLLddddddddddLLL | GTCCTTCTAATCC | 771-786 |
| | | | CGT | |
| 104 | 3-10-3 | LLLddddddddddLLL | GCTTTTCGGACTG | 784-799 |
| | | | GTC | |
| 105 | 3-10-3 | LLLddddddddddLLL | TGCTTTTCGGACT | 785-800 |
| | | | GGT | |
| 106 | 3-10-3 | LLLddddddddddLLL | CTGCTTTTCGGAC | 786-801 |
| | | | TGG | |
| 107 | 3-10-3 | LLLddddddddddLLL | TCCGTGCCCTGAA | 811-826 |
| | | | CTT | |
| 108 | 3-10-3 | LLLddddddddddLLL | CTCCGTGCCCTGA | 812-827 |
| | | | ACT | |
| 109 | 3-10-3 | LLLddddddddddLLL | AGTAGTCCTCCGT | 819-834 |
| | | | GCC | |
| 110 | 3-10-3 | LLLddddddddddLLL | GCAGTAGTCCTCC | 821-836 |
| | | | GTG | |
| 111 | 3-10-3 | LLLddddddddddLLL | TGCAGTAGTCCTC | 822-837 |
| | | | CGT | |
| 112 | 3-10-3 | LLLddddddddddLLL | CATCTGCCTTTCA | 845-860 |
| | | | CGC | |
| 113 | 3-10-3 | LLLddddddddddLLL | CCGCAATGCCCAC | 1084-1099 |
| | | | ACA | |
| 114 | 3-10-3 | LLLddddddddddLLL | ATCTGCCGCAATG | 1089-1104 |
| | | | CCC | |
| 115 | 3-10-3 | LLLddddddddddLLL | AATCTGCCGCAAT | 1090-1105 |
| | | | GCC | |
| 116 | 3-10-3 | LLLddddddddddLLL | GTTACAGCGACGA | 1159-1174 |
| | | | CAA | |
| 117 | 3-10-3 | LLLddddddddddLLL | AGTTACAGCGACG | 1160-1175 |
| | | | ACA | |
| 118 | 3-10-3 | LLLddddddddddLLL | CAGTTACAGCGAC | 1161-1176 |
| | | | GAC | |
| 119 | 3-10-3 | LLLddddddddddLLL | AGACAGTTACAGC | 1164-1179 |
| | | | GAC | |
| 120 | 3-10-3 | LLLddddddddddLLL | CCCCGGTTATTTA | 1244-1259 |
| | | | GGT | |
| 121 | 3-10-3 | LLLddddddddddLLL | TCCCCGGTTATTT | 1245-1260 |
| | | | AGG | |
| 122 | 3-10-3 | LLLddddddddddLLL | CTCCCCGGTTATT | 1246-1261 |
| | | | TAG | |
| 123 | 3-10-3 | LLLddddddddddLLL | TGAGACACGTGCG | 68-83 |
| | | | GGG | |
| 124 | 3-10-3 | LLLddddddddddLLL | CTGAGACACGTGC | 69-84 |
| | | | GGG | |
| 125 | 3-10-3 | LLLddddddddddLLL | CCTGAGACACGTG | 70-85 |
| | | | CGG | |
| 126 | 3-10-3 | LLLddddddddddLLL | AGTGGCAGGTCGG | 362-377 |
| | | | GTT | |
| 127 | 3-10-3 | LLLddddddddddLLL | GAAGTGGCAGGTC | 364-379 |
| | | | GGG | |
| 128 | 3-10-3 | LLLddddddddddLLL | CTACTCCTTGGAA | 374-389 |
| | | | GTG | |
| 129 | 3-10-3 | LLLddddddddddLLL | AAATTGGGGTAGG | 415-430 |
| | | | GCT | |
| 130 | 3-10-3 | LLLddddddddddLLL | AGGTAAAGAGAG | 198-213 |
| | | | TCAG | |
| 131 | 3-9-3 | MMMdddddddddMMM | AACACGTCTATAC | Control |
| | | | GC | gapmer |
| 132 | 3-9-3 | LLLddddddddddLLL | AACACGTCTATAC | Control |
| | | | GC | gapmer |

As IL-1β-eRNA is a lncRNA that regulates IL-1β transcription, the gapmer compounds were analyzed for their effect on IL-1β transcription by quantitative real-time PCR. Similarly, the effect of the gapmer compounds on cytotoxicity and on Toll-like receptor (TLR) signaling activation were analyzed by assaying for the gene transcription of TNFRSF10B and secreted embryonic alkaline phosphatase (SEAP), respectively. Data are averages from four replicates in which THP1 cells were treated with the gapmer compounds of Table 1.

Table 2. shows the fold change of IL1β, TNFRSF10B, and SEAP gene expression in THP1 cells in the presence of the gapmer compounds in Table 1. If present, “N.D.” indicates “no data”. Data are normalized by the expression of housekeeping gene RPL37A and represented as fold change relative to negative control (SEQ ID NO. 131 served as a negative control for MOE gapmers, and SEQ ID NO. 132 served as a negative control for LNA gapmers). An expression value <1.0 means that the transcription of that gene was inhibited, and an expression value >1.0 means that the transcription of that gene is induced. For example, a value of 0.25 means that gene transcription was inhibited by 75%.

TABLE 2

| SEQ | IL1B | TNFRSF10B | SEAP |
|---|---|---|---|
| ID NO. | EXPRESSION | EXPRESSION | EXPRESSION |
| | 1.033 | 0.985 | 0.817 |
| 3 | 0.953 | 1.012 | 1.251 |
| 4 | 1.684 | 1.052 | 2.161 |
| 5 | 0.538 | 0.978 | 0.736 |
| 6 | 1.332 | 0.874 | 1.459 |
| 7 | 1.249 | 0.917 | 1.580 |
| 8 | 1.164 | 1.144 | 1.656 |
| 9 | 0.512 | 0.949 | 1.166 |
| 10 | 1.253 | 0.889 | 1.150 |
| 11 | 0.834 | 0.809 | 0.756 |
| 12 | 1.048 | 0.837 | 0.862 |
| 13 | 0.839 | 0.564 | 1.022 |
| 14 | 0.579 | 0.560 | 1.143 |
| 15 | 2.372 | 1.272 | 2.112 |
| 16 | 1.537 | 0.940 | 2.144 |
| 17 | 2.189 | 1.212 | 2.268 |
| 18 | 1.565 | 1.200 | 1.721 |
| 19 | 0.696 | 0.823 | 1.212 |
| 20 | 1.065 | 0.513 | 0.688 |
| 21 | 1.154 | 0.871 | 1.359 |
| 22 | 0.895 | 0.999 | 1.472 |
| 23 | 1.588 | 1.154 | 1.592 |
| 24 | 1.412 | 1.014 | 1.421 |
| 25 | 0.717 | 0.670 | 1.262 |
| 26 | 1.335 | 0.934 | 1.381 |
| 27 | 1.003 | 0.959 | 1.209 |
| 28 | 0.714 | 0.655 | 1.230 |
| 29 | 1.328 | 1.181 | 1.550 |
| 30 | 0.953 | 1.686 | 1.212 |
| 31 | 0.921 | 1.400 | 1.087 |
| 32 | 1.436 | 1.712 | 1.849 |
| 33 | 1.876 | 1.386 | 1.377 |
| 34 | 0.805 | 1.248 | 0.963 |
| 35 | 1.275 | 1.321 | 1.056 |
| 36 | 0.919 | 1.597 | 1.128 |
| 37 | 1.166 | 1.679 | 1.088 |
| 38 | 1.051 | 1.584 | 1.197 |
| 39 | 0.684 | 0.869 | 0.799 |
| 40 | 1.340 | 1.683 | 1.301 |
| 41 | 3.188 | 1.765 | 1.640 |
| 42 | 0.718 | 1.165 | 1.203 |
| 43 | 1.007 | 0.691 | 0.813 |
| 44 | 1.056 | 1.173 | 0.891 |
| 45 | 0.637 | 0.739 | 0.627 |
| 46 | 0.770 | 0.589 | 0.744 |
| 47 | 0.725 | 0.622 | 0.702 |
| 48 | 0.443 | 0.519 | 0.329 |
| 49 | 0.989 | 0.452 | 0.645 |
| 50 | 0.430 | 0.323 | 0.308 |
| 51 | 0.467 | 0.461 | 0.303 |
| 52 | 0.689 | 1.221 | 0.480 |
| 53 | 1.234 | 1.241 | 1.173 |
| 54 | 0.948 | 3.123 | 1.169 |
| 55 | 0.913 | 2.387 | 0.921 |
| 56 | 1.193 | 2.853 | 0.933 |
| 57 | 1.310 | 1.946 | 1.213 |
| 58 | 0.576 | 0.211 | 0.581 |
| 59 | 1.030 | 1.387 | 1.196 |
| 60 | 0.768 | 2.200 | 0.580 |
| 61 | 1.020 | 1.741 | 0.998 |
| 62 | 1.591 | 1.488 | 2.216 |
| 63 | 0.731 | 1.529 | 0.933 |
| 64 | 0.549 | N.D. | 0.625 |
| 65 | 0.471 | 0.929 | 0.394 |
| 66 | 0.736 | 0.976 | 0.991 |
| 67 | 0.721 | 0.955 | 1.001 |
| 68 | 0.505 | 0.417 | 0.531 |
| 69 | 0.358 | 0.623 | 0.996 |
| 70 | 0.761 | 1.079 | 1.131 |
| 71 | 1.036 | 1.691 | 2.954 |
| 72 | 0.977 | 1.785 | 1.930 |
| 73 | 0.768 | N.D. | 1.463 |
| 74 | 1.172 | 1.470 | 0.537 |
| 75 | 1.598 | 2.364 | 0.973 |
| 76 | 0.776 | 1.973 | 0.669 |
| 77 | 1.101 | 1.123 | 0.784 |
| 78 | 1.225 | 1.296 | 1.092 |
| 79 | 1.386 | 1.454 | 0.960 |
| 80 | 1.249 | 2.488 | 1.052 |
| 81 | 1.006 | 1.564 | 1.471 |
| 82 | 2.171 | 1.898 | 1.141 |
| 83 | 1.098 | 2.510 | 0.974 |
| 84 | 0.402 | 0.906 | 0.563 |
| 85 | 1.362 | 1.489 | 0.667 |
| 86 | 0.769 | 1.774 | 0.374 |
| 87 | 1.134 | 1.129 | 0.416 |
| 88 | 1.618 | 0.854 | 1.119 |
| 89 | 1.037 | 1.641 | 1.345 |
| 90 | 1.156 | 2.067 | 1.217 |
| 91 | 1.252 | 1.524 | 0.961 |
| 92 | 0.758 | N.D. | 0.859 |
| 93 | 0.662 | N.D. | 1.187 |
| 94 | 1.715 | N.D. | 1.444 |
| 95 | 1.880 | N.D. | 2.303 |
| 96 | 3.168 | N.D. | 1.771 |
| 97 | 2.174 | N.D. | 1.665 |
| 98 | 1.515 | N.D. | 1.118 |
| 99 | 1.624 | N.D. | 1.568 |
| 100 | 1.088 | N.D. | 1.098 |
| 101 | 1.142 | N.D. | 1.046 |
| 102 | 5.744 | N.D. | 2.658 |
| 103 | 1.775 | N.D. | 1.025 |
| 104 | 0.659 | N.D. | 0.785 |
| 105 | 1.325 | N.D. | 1.231 |
| 106 | 1.143 | N.D. | 1.083 |
| 107 | 1.118 | N.D. | 1.188 |
| 108 | 1.211 | N.D. | 1.132 |
| 109 | 0.952 | N.D. | 0.977 |
| 110 | 1.080 | N.D. | 0.837 |
| 111 | 1.204 | N.D. | 1.216 |
| 112 | 0.913 | N.D. | 0.714 |
| 113 | 1.170 | N.D. | 0.802 |
| 114 | 0.853 | N.D. | 0.725 |
| 115 | 1.189 | N.D. | 0.909 |
| 116 | 1.217 | N.D. | 0.848 |
| 117 | 1.399 | N.D. | 0.942 |
| 118 | 1.309 | N.D. | 1.254 |
| 119 | 4.325 | N.D. | 1.513 |
| 120 | 1.031 | N.D. | 1.096 |
| 121 | 0.654 | N.D. | 0.580 |
| 122 | 0.387 | N.D. | 0.345 |
| 123 | 0.947 | N.D. | 0.556 |
| 124 | 1.298 | N.D. | 1.204 |
| 125 | 1.745 | N.D. | 1.047 |
| 126 | 1.199 | N.D. | 0.847 |
| 127 | 1.538 | N.D. | 0.511 |
| 128 | 1.511 | N.D. | 0.986 |
| 129 | 0.631 | N.D. | 0.727 |
| 130 | 0.962 | 1.133 | 0.998 |

The data in Table 2 show that gapmers SEQ ID NO. 48, 50, 51, 64, 65, and 122 demonstrated at least 50% inhibition of human IL1β expression in this assay.

Based on the screening results in Table 1 and 2, a group of chimeric phosphorothioate gapmer compounds that efficiently targeted human IL-1β eRNA (SEQ ID NO. 1) was synthesized. Table 3 provides the configuration, chemical modification, and sequence of the chimeric phosphorothioate gapmer compounds of the newly synthesized chimeric phosphorothioate gapmer compounds. Abbreviations of the nucleoside modification in Table 3: M=2′-methoxyethyl (2′-MOE) modified nucleoside; L=locked nucleic acid (LNA) modified nucleoside (cMe); d=2′-deoxynucleosides; 2′Md=2′OMe modified deoxynucleoside.

TABLE 3

| | | | | Complementary |
|---|---|---|---|---|
| | | | | position to |
| | | | | human |
| SEQ | Con- | Nucleoside | Sequence of | IL-1β eRNA |
| ID NO. | figuration | Modification | gapmer compound | (SEQ ID NO. 1) |
| 133 | 5-10-5 | MMMMMddddddddddMMM | GACACGTGCGGGG | 61-80 |
| | | MM | AAATTCT | |
| 134 | 6-8-6 | MMMMMMddddddddMMM | GACACGTGCGGGG | 61-80 |
| | | MMM | AAATTCT | |
| 135 | 7-6-7 | MMMMMMMddddddMMM | GACACGTGCGGGG | 61-80 |
| | | MMMM | AAATTCT | |
| 136 | 4-10-4 | MMMMddddddddddMMMM | GACACGTGCGGGG | 63-80 |
| | | | AAATT | |
| 137 | 5-8-5 | MMMMMddddddddMMMM | GACACGTGCGGGG | 63-80 |
| | | M | AAATT | |
| 138 | 4-8-4 | MMMMddddddddMMMM | GACACGTGCGGGG | 65-80 |
| | | | AAA | |
| 139 | 5-6-5 | MMMMMddddddMMMMM | GACACGTGCGGGG | 65-80 |
| | | | AAA | |
| 140 | 3-10-4 | LLLddddddddddLLLL | GACACGTGCGGGG | 64-80 |
| | | | AAAT | |
| 141 | 5-8-4 | LLLLLddddddddLLLL | GACACGTGCGGGG | 64-80 |
| | | | AAAT | |
| 69 | 3-10-3 | LLLddddddddddLLL | GACACGTGCGGGG | 65-80 |
| | | | AAA | |
| 142 | 5-7-4 | LLLLLdddddddLLLL | GACACGTGCGGGG | 65-80 |
| | | | AAA | |
| 143 | 3-10-3 | LLLdd2′MddddddddLLL | GACACGTGCGGGG | 65-80 |
| | | | AAA | |
| 144 | 5-10-5 | MMMMMddddddddddMM | ACAGACGAGTTA | 487-506 |
| | | MMM | CTCCCAGG | |
| 145 | 6-8-6 | MMMMMMddddddddMMM | ACAGACGAGTTA | 487-506 |
| | | MMM | CTCCCAGG | |
| 146 | 7-6-7 | MMMMMMMddddddMMM | ACAGACGAGTTA | 487-506 |
| | | MMMM | CTCCCAGG | |
| 147 | 4-10-4 | MMMMddddddddddMMM | ACAGACGAGTTA | 489-506 |
| | | M | CTCCCA | |
| 148 | 5-8-5 | MMMMMddddddddMMMM | ACAGACGAGTTA | 489-506 |
| | | M | CTCCCA | |
| 149 | 4-8-4 | MMMMddddddddMMMM | CAGACGAGTTAC | 490-505 |
| | | | TCCC | |
| 150 | 5-6-5 | MMMMMddddddMMMMM | CAGACGAGTTAC | 490-505 |
| | | | TCCC | |
| 151 | 3-10-4 | LLLddddddddddLLLL | ACAGACGAGTTA | 490-506 |
| | | | CTCCC | |
| 152 | 5-8-4 | LLLLLddddddddLLLL | ACAGACGAGTTA | 490-506 |
| | | | CTCCC | |
| 84 | 3-10-3 | LLLddddddddddLLL | CAGACGAGTTAC | 490-505 |
| | | | TCCC | |
| 153 | 5-7-4 | LLLLLdddddddLLLL | CAGACGAGTTAC | 490-505 |
| | | | TCCC | |
| 154 | 3-10-3 | LLLdd2′MddddddddLLL | CAGACGAGTTAC | 490-505 |
| | | | TCCC | |
| 51 | 5-10-5 | MMMMMddddddddddMM | GTTCTTCTGGATC | 1278-1297 |
| | | MMM | CATCTCT | |
| 155 | 6-8-6 | MMMMMMddddddddMMM | GTTCTTCTGGATC | 1278-1297 |
| | | MMM | CATCTCT | |
| 156 | 7-6-7 | MMMMMMMddddddMMM | GTTCTTCTGGATC | 1278-1297 |
| | | MMMM | CATCTCT | |
| 157 | 4-10-4 | MMMMddddddddddMMM | GTTCTTCTGGATC | 1280-1297 |
| | | M | CATCT | |
| 158 | 5-8-5 | MMMMMddddddddMMMM | GTTCTTCTGGATC | 1280-1297 |
| | | M | CATCT | |
| 159 | 4-8-4 | MMMMddddddddMMMM | GTTCTTCTGGATC | 1282-1297 |
| | | | CAT | |
| 160 | 5-6-5 | MMMMMddddddMMMMM | GTTCTTCTGGATC | 1282-1297 |
| | | | CAT | |
| 161 | 3-10-4 | LLLddddddddddLLLL | GTTCTTCTGGATC | 1281-1297 |
| | | | CATC | |
| 162 | 5-8-4 | LLLLLddddddddLLLL | GTTCTTCTGGATC | 1281-1297 |
| | | | CATC | |
| 163 | 3-10-3 | LLLddddddddddLLL | GTTCTTCTGGATC | 1282-1297 |
| | | | CAT | |
| 164 | 5-7-4 | LLLLLdddddddLLLL | GTTCTTCTGGATC | 1282-1297 |
| | | | CAT | |
| 165 | 3-10-3 | LLLdd2′MddddddddLLL | GTTCTTCTGGATC | 1282-1297 |
| | | | CAT | |
| 50 | 5-10-5 | MMMMMddddddddddMM | TACAGCGACGAC | 1153-1172 |
| | | MMM | AATAATAA | |
| 166 | 6-8-6 | MMMMMMddddddddMMM | TACAGCGACGAC | 1153-1172 |
| | | MMM | AATAATAA | |
| 167 | 7-6-7 | MMMMMMMddddddMMM | TACAGCGACGAC | 1153-1172 |
| | | MMMM | AATAATAA | |
| 168 | 4-10-4 | MMMMddddddddddMMM | TACAGCGACGAC | 1155-1172 |
| | | M | AATAAT | |
| 169 | 5-8-5 | MMMMMddddddddMMMM | TACAGCGACGAC | 1155-1172 |
| | | M | AATAAT | |
| 170 | 4-8-4 | MMMMddddddddMMMM | TACAGCGACGAC | 1157-1172 |
| | | | AATA | |
| 171 | 5-6-5 | MMMMMddddddMMMMM | TACAGCGACGAC | 1157-1172 |
| | | | AATA | |
| 172 | 3-10-4 | LLLddddddddddLLLL | TACAGCGACGAC | 1156-1172 |
| | | | AATAA | |
| 173 | 5-8-4 | LLLLLddddddddLLLL | TACAGCGACGAC | 1156-1172 |
| | | | AATAA | |
| 174 | 3-10-3 | LLLddddddddddLLL | TACAGCGACGAC | 1157-1172 |
| | | | AATA | |
| 175 | 5-7-4 | LLLLLdddddddLLLL | TACAGCGACGAC | 1157-1172 |
| | | | AATA | |
| 176 | 3-10-3 | LLLdd2′MddddddddLLL | TACAGCGACGAC | 1157-1172 |
| | | | AATA | |

The chimeric phosphorothioate gapmer compounds in Table 3 were analyzed for their effect on IL1β, TNFRSF10B, and SEAP transcription in THP1 cells by quantitative real-time PCR. Table 4 shows gene expression of IL1B, TNFRSF10B, and SEAP by chimeric phosphorothioate gapmers SEQ ID NOs 50, 51, 69, and 84 that target human IL-1β eRNA sequence (SEQ ID NO. 1). Data are normalized by the expression of housekeeping gene RPL37A and represented as fold change relative to negative control (SEQ ID NO. 131 served as a negative control for MOE gapmers, and SEQ ID NO. 132 served as a negative control for LNA gapmers). An expression value <1.0 means that the transcription of that gene was inhibited, and an expression value >1.0 means that the transcription of that gene is induced. For example, a value of 0.25 means that gene transcription was inhibited by 75%.

TABLE 4

| SEQ | IL1B | TNFRSF10B | SEAP |
|---|---|---|---|
| ID NO. | EXPRESSION | EXPRESSION | EXPRESSION |
| 133 | 1.436 | 1.238 | 1.394 |
| 134 | 1.608 | 1.291 | 1.742 |
| 135 | 1.210 | 1.337 | 1.564 |
| 136 | 1.475 | 1.336 | 1.532 |
| 137 | 1.340 | 1.557 | 1.283 |
| 138 | 1.981 | 0.988 | 1.604 |
| 139 | 1.465 | 1.222 | 1.295 |
| 140 | 1.002 | 1.245 | 1.214 |
| 141 | 0.733 | 0.735 | 0.624 |
| 69 | 1.404 | 1.649 | 1.085 |
| 142 | 0.373 | 0.553 | 0.393 |
| 143 | 1.182 | 1.268 | 1.373 |
| 144 | 0.766 | 0.693 | 0.669 |
| 145 | 0.768 | 0.588 | 0.882 |
| 146 | 0.500 | 0.217 | 0.700 |
| 147 | 0.658 | 0.520 | 0.630 |
| 148 | 0.377 | 0.446 | 0.579 |
| 149 | 0.712 | 0.703 | 0.853 |
| 150 | 0.575 | 0.491 | 0.791 |
| 151 | 0.509 | 0.602 | 0.738 |
| 152 | 0.884 | 0.692 | 1.023 |
| 84 | 0.903 | 0.519 | 1.387 |
| 153 | 1.168 | 1.572 | 0.828 |
| 154 | 0.825 | 1.587 | 0.797 |
| 51 | 0.982 | 1.066 | 0.776 |
| 155 | 1.048 | 1.271 | 1.105 |
| 156 | 0.826 | 1.363 | 0.797 |
| 157 | 1.378 | 1.571 | 0.977 |
| 158 | 1.642 | 1.660 | 0.989 |
| 159 | 1.158 | 1.302 | 1.205 |
| 160 | 1.789 | 1.806 | 1.270 |
| 161 | 1.317 | 1.342 | 1.179 |
| 162 | 1.295 | 1.403 | 1.039 |
| 163 | 1.114 | 1.491 | 1.124 |
| 164 | 1.541 | 0.833 | 1.223 |
| 165 | 1.198 | 2.021 | 1.617 |
| 50 | 0.580 | 0.711 | 1.167 |
| 166 | 0.704 | 0.960 | 1.131 |
| 167 | 0.731 | 0.708 | 1.110 |
| 168 | 0.745 | 0.783 | 1.113 |
| 169 | 0.828 | 0.759 | 1.055 |
| 170 | 0.689 | 0.788 | 1.040 |
| 171 | 0.662 | 0.391 | 1.281 |
| 172 | 0.635 | 0.979 | 0.963 |
| 173 | 1.021 | 0.572 | 0.761 |
| 174 | 0.468 | 1.430 | 1.312 |
| 175 | 0.579 | 1.774 | 0.619 |
| 176 | 0.856 | 2.101 | 0.787 |

Based on the screening data in Table 1-4, three regions on the target IL-1β-eRNA sequence (SEQ ID NO. 1) were found for gapmers SEQ ID NOs: 64, 65, 68, 69, 50, 25, 51, 121, 122, and 131. Table 5 provides the positions of IL-1β eRNA target regions (A, B, and C) in human IL-1β eRNA sequence (SEQ ID NO.1) and the average % inhibition of IL1B gene expression by the gapmers targeted to individual regions.

TABLE 5

| IL-1β eRNA | Target Region in | | AVERAGE |
|---|---|---|---|
| Target Region | human IL-1β eRNA | ASOs: SEQ ID | IL1B |
| ID | (SEQ ID NO. 1) | NO. | EXPRESSION |
| A | 58-80 | 64, 65, 68, 69 | 53.9% |
| B | 1153-1172 | 50, 174 | 55.1% |
| C | 1245-1297 | 25, 51, 121, 122 | 44.4% |

All of the gapmers targeted to Regions A, B, and C of IL-1β eRNA. at positions of 58-80, 1153-1172, and 1245-1297, respectively, demonstrated more than 40% inhibition of IL1β gene expression.

### Example 2: IL-1β eRNA Inhibition in LPS Stimulated CD14 Positive Monocytes

Percoll monocytes from healthy donors (n=3) were seeded in serum-free RPMI 1640 containing GlutaMAX (Gibco), 10% FBS (Gibco) and 1% Pen/Strep (Gibco) in a 24 well plate (Sarstedt) at 1×106 cells/well and incubated at 37° C. at 5% CO₂ for 1 hour. Plated cells were washed with serum-free RPMI and treated with RPMI+10% pooled human serum containing gapmer ASOs SEQ ID NO. 69 or SEQ ID NO. 132 gapmer compounds and incubated at 37° C. at 5% CO₂ overnight. Each treatment was conducted in duplicate. Plated cells were then treated with 10 ng/ml Ultrapure LPS (Invivogen) and incubated at 37° C. at 5% CO₂ overnight. Gapmer-treated cells were then centrifuged at 400 rpm for 5 min at room temperature and RNA was isolated using the MagMAX RNA Total RNA Isolation Kit (Thermo Fisher Scientific) in accordance with the manufacturer's recommended protocols. Total RNA was quantified using the NanoDrop® (Thermo Fisher Scientific) manufacturer's recommended protocols. Total RNA underwent a reverse transcriptase reaction (RT) using an iScript cDNA synthesis kit (Bio-Rad) according to the manufacturer's recommended reagents and protocols to produce complementary DNA to be used as substrate for quantitative real-time polymerase chain reaction (RT-qPCR). Quantification of target RNA levels was conducted by RT-qPCR with SsoAdvanced Universal SYBR Green according to the manufacturer's recommended protocols using a CFX Real-time PCR detection system (Bio-Rad). The target quantities obtained by RT-qPCR were normalized using the expression of a stably expressed housekeeping gene, RPL37A.

Table 6 provides the fold change of IL1B gene expression in LPS-treated monocytes (n=3 donors) in the presence of the gapmer compounds (SEQ ID NO. 69). Data were normalized by the expression of housekeeping gene RPL37A and represented as fold change relative to LPS-treated monocytes cells with control gapmer compound (SEQ ID NO. 132). An expression value <1.0 means that the transcription of that gene was inhibited, and an expression value >1.0 means that the transcription of that gene was induced. For example, a value of 0.25 means that gene transcription was inhibited by 75%.

TABLE 6

| | AVERAGE SEQ ID | |
|---|---|---|
| SEQ ID NO. | 1 EXPRESSION | IL1B EXPRESSION |
| 69 | 0.25 | 0.230453 |
| 132 | 1 | 1 |

### Example 3: IL-1β eRNA Inhibition in Prostatic Adenocarcinoma Cell Line, PC-3

PC-3 cells (ATCC) were seeded at 6×104 per well in a 24-well plate (Sarstedt) RPMI 1640 containing GlutaMAX (Gibco), 10% FBS (Gibco) and 1% Pen/Strep (Gibco) and gapmer compounds SEQ ID NO. 50. Gapmer compounds were added at a 5 uM (low) or 30 uM (high) dose. Each treatment was conducted in quadruplicate. Plated cells were incubated at 37° C. at 5% CO2 for 24 hours. Gapmer-treated cells were then centrifuged at 400 rpm for 5 min at room temperature and RNA was isolated using the MagMAX RNA miRVana Total RNA Isolation Kit (Thermo Fisher Scientific) in accordance with the manufacturer's recommended protocols. Total RNA was quantified using the NanoDrop® (Thermo Fisher Scientific) according to manufacturer's protocols. Total RNA underwent a reverse transcriptase reaction (RT) using an iScript cDNA synthesis kit (Bio-Rad) according to the manufacturer's recommended reagents and protocols to produce complementary DNA to be used as substrate for quantitative real-time polymerase chain reaction (RT-qPCR). Quantification of target RNA levels was conducted by RT-qPCR with SsoAdvanced Universal SYBR Green according to the manufacturer's recommended protocols using a CFX Real-time PCR detection system (Bio-Rad). The target quantities obtained by RT-qPCR were normalized using the expression of a stably expressed housekeeping gene, RPL37A.

Table 7 shows the fold change of IL1B gene expression in PC-3 cells in the presence of the gapmer compounds. Data were normalized by the expression of housekeeping gene RPL37A and represented as fold change relative to untreated PC-3 cells. An expression value <1.0 means that the transcription of that gene is inhibited, and an expression value >1.0 means that the transcription of that gene is induced. For example, a value of 0.25 means that gene transcription was inhibited by 75%.

TABLE 7

| SEQ ID NO. | GAPMER DOSAGE | IL1B EXPRESSION |
|---|---|---|
| 69 | LOW | 0.76 |
| 69 | HIGH | 0.69 |

### Example 4: IL-1β eRNA Inhibition in PBMCs Treated with LPS and R848

Peripheral blood mononuclear cells (PBMCs) from healthy donors (n=3) with an average age of 56.3±16.8 years were purchased cryopreserved from CTL Europe. Cryopreserved PBMCs were thawed in pre-warmed Wash Media (RPMI (Dutch modification)+20% FBS+2 mM GlutaMAX+1 mM Sodium pyruvate+1% Pen/Strep). PBMCs were then washed in pre-warmed Wash Media three times before centrifugation in pre-warmed Wash Media at 500 rpm for 10 min at room temperature. Centrifugation was repeated twice before resuspending cells in pre-warmed Culture Media (RPMI (Dutch modification)+10% FBS+2 mM GlutaMAX+1 mM Sodium pyruvate+1% Pen/Strep). Next, cells were counted and seeded at approximately 1×105 cells were added into each well of a 96-well U-bottom tissue culture plate (CellSTAR) containing a TLR cocktail containing 100 ng/mL Ultrapure LPS (Invivogen) and 500 ng/mL Resiquimod (R848, Invivogen), with or without gapmer compounds (SEQ ID 50). Gapmer compounds were added at a final concentration of 5 uM. Each treatment was conducted in duplicate and plated cells were incubated at 37° C. at 5% CO2 for 48 hours. Plated cells were then centrifuged at 400 rpm for 5 min at room temperature and RNA was isolated using MagMAX RNA Total RNA Isolation Kit (Thermo Fisher Scientific) in accordance with the manufacturer's recommended protocols. Total RNA was then quantified using the NanoDrop® (Thermo Fisher Scientific) according to manufacturer's recommended protocols. Total RNA then underwent a reverse transcriptase reaction (RT) using an iScript cDNA synthesis kit (Bio-Rad) according to the manufacturer's recommended reagents and protocols to produce complementary DNA. Complementary DNA was used as substrate for quantitative real-time polymerase chain reaction (RT-qPCR) using SsoAdvanced Universal SYBR Green in accordance with manufacturer's recommended protocols. Quantification of target RNA levels was conducted by RT-qPCR using a CFX Real-time PCR detection system (Bio-Rad). The target quantities obtained by RT-qPCR were normalized using the expression of a stably expressed housekeeping gene, RPL37A.

Table 8 shows the fold change of IL1B gene expression in TLR-stimulated PBMCs in the presence of the gapmer compound (SEQ ID NO. 50). Data were normalized by expression of housekeeping gene RPL37A and represented as a fold change relative to PBMCs treated with TLR cocktails without gapmer compounds. An expression value <1.0 means that the transcription of that gene was inhibited, and an expression value >1.0 means that the transcription of that gene was induced. For example, a value of 0.25 means that gene transcription was inhibited by 75%.

TABLE 8

| SEQ ID NO. | DONOR ID | IL1B EXPRESSION |
|---|---|---|
| 50 | 1 | 0.76 |
| 50 | 2 | 0.82 |
| 50 | 3 | 0.82 |

### Example 5: IL-1β eRNA Inhibition LPS Stimulated Mouse Macrophage Cell Line, RAW264.7

This example provides cross-reacting different species (human and mouse) gapmer compounds to enable in vivo testing in murine models. Human (SEQ ID NO. 1) and murine (SEQ ID NO. 2) IL-1β eRNA target sequences were compared for regions of homology but none were found to be as long as 20 nucleotides. Three gapmer antisense sequences which were complementary to either human and murine IL-1β eRNA and which had no more than 1 mismatch to murine IL-1β eRNA were designed. These gapmers were designed to function in both in vitro models with human cells and in murine in vivo models. However, the relative antisense efficacy may not be equal for these two forms because of imperfect homology to one IL-1β eRNA or the other.

TABLE 9A

| | | | | Complementary |
|---|---|---|---|---|
| | | | | position to |
| | | | | human |
| | | | | IL-1β eRNA |
| SEQ | Con- | | Sequence of | lnc RNA of SEQ |
| ID NO. | figuration | Chemimstry | gapmer compound | ID NO: 2 |
| 106 | 5-10-5 | MMMMMddddddddddMMM | CTCACCCCCATAC | 384-403 |
| | | MM | ATCTCTT | |

RAW 264.7 cells (ATCC) were seeded at 2.5×105 per well in a 24-well plate (Sarstedt) in DMEM (Gibco) containing 10% FBS (Fisher Scientific) and 1% Pen/Strep (Gibco). Plated cells were incubated at 37° C. at 5% CO2 for 24 hours. Cells were then treated with 10 ng/ml of Ultrapure LPS (Invitrogen) and incubated at 37° C. at 5% CO2 for 24 hours. Each treatment was conducted in quadruplicate. Cells were then treated with 5 micromolar (low), 10 micromolar (med) and 30 micromolar (high) gapmer compounds (SEQ ID NO. 106).

Table 9B displays the fold change of IL1beta gene expression in LPS-stimulated RAW 264.7 cells in the presence of the gapmer compounds (SEQ ID NO. 106). Data were normalized by the expression of housekeeping gene RPL37A and represented as fold change relative to RAW 264.7 cells treated with LPS cocktails without gapmer compounds. An expression value <1.0 means that the transcription of that gene is inhibited, and an expression value >1.0 means that the transcription of that gene is induced. For example, a value of 0.25 means that gene transcription was inhibited by 75%.

TABLE 9B

| SEQ ID NO. | Gapmer Dosage | IL-1β Expression |
|---|---|---|
| 104 | Low | 0.82 |
| 105 | Med | 0.65 |
| 106 | High | 0.62 |

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A gapmer compound that inhibits IL-1β eRNA transcription comprising from about 14 to about 25 nucleotide bases having (3′ to 5′) a 3″ wing region comprising from 3 to 7 chemically modified RNA bases, a gap region comprising from at least 8 DNA bases, and a 5″ wing region comprising from 3 to 7 chemically modified RNA bases, and wherein the gapmer is substantially complementary to a 14-25 base region on IL-1β eRNA (SEQ ID NO. 1).

2. The gapmer compound that inhibits IL-1β transcription of claim 1, wherein the gapmer nucleotides are each linked by phosphorothiolate (P═S) internucleotide bonds throughout the gapmer; and wherein the modified nucleotide base modifications are selected from the group consisting of a 2′-methoxyethyl (MOE) modification, a locked nucleic acid (LNA) modification, a 2′F-ANA modification, a 2′-O-methoxyethyl (2′OMe) modification, and combinations thereof.

3. A gapmer compound that that inhibits acute inflammatory IL-1β gene transcription regulated by IL-1β eRNA, comprising: (a) a 5′ wing sequence having from about 3 to about 7 wing modified nucleotide bases; (b) a central gap region sequence having from about 8 to about 15 2′ deoxynucleotides; and (c) a 3′ wing sequence having from about 3 to about 7 wing modified nucleotide bases; wherein the gapmer sequence is complementary to Region A of IL-1β eRNA (SEQ ID NO. 1) bases 58 to 80.

4. The gapmer compound that that inhibits acute inflammatory IL-1β gene transcription regulated by IL-1β eRNA of claim 3, wherein the modified nucleotide base modifications are selected from the group consisting of a 2′-methoxyethyl (MOE) modification, a locked nucleic acid (LNA) modification, a 2′F-ANA modification, a 2′-O-methoxyethyl (2′OMe) modification, and combinations thereof.

5. The gapmer compound that inhibits acute inflammatory IL-1β gene transcription regulated by IL-1β eRNA of claim 3, wherein the gapmer compound that is complementary to Region A is selected from the group consisting of SEQ ID NOs. 64, 65, 68, 69 and combinations thereof.

6. A gapmer compound that that inhibits acute inflammatory IL-1β gene transcription regulated by IL-1β eRNA, comprising: (a) a 5′ wing sequence having from about 3 to about 7 wing modified nucleotide bases; (b) a central gap region sequence having from about 8 to about 15 2′ deoxynucleotides; and (c) a 3′ wing sequence having from about 3 to about 7 wing modified nucleotide bases; wherein the gapmer sequence is complementary to Region B of IL-1β eRNA (SEQ ID NO. 1) base 1153 to base 1172.

7. The gapmer compound that that inhibits acute inflammatory IL-1 gene transcription regulated by IL-1β eRNA of claim 6, wherein the modified nucleotide base modifications are selected from the group consisting of a 2′-methoxyethyl (MOE) modification, a locked nucleic acid (LNA) modification, a 2′F-ANA modification, a 2′-O-methoxyethyl (2′OMe) modification, and combinations thereof.

8. The gapmer compound that inhibits acute inflammatory IL-1β gene transcription regulated by IL-1β eRNA of claim 6, wherein the gapmer compound that is complementary to Region B is SEQ ID NOs. 50, 174 and combinations thereof.

9. A gapmer compound that that inhibits acute inflammatory IL-1β gene transcription regulated by IL-1β eRNA, comprising: (a) a 5′ wing sequence having from about 3 to about 7 wing modified nucleotide bases; (b) a central gap region sequence having from about 8 to about 15 2′ deoxynucleotides; and (c) a 3′ wing sequence having from about 3 to about 7 wing modified nucleotide bases; wherein the gapmer sequence is complementary to Region C of IL-1β eRNA (SEQ ID NO. 1) base 1245 to base 1297.

10. The gapmer compound that that inhibits acute inflammatory IL-1β gene transcription regulated by IL-1β eRNA of claim 9, wherein the modified nucleotide base modifications are selected from the group consisting of a 2′-methoxyethyl (MOE) modification, a locked nucleic acid (LNA) modification, a 2′F-ANA modification, a 2′-O-methoxyethyl (2′OMe) modification, and combinations thereof.

11. The gapmer compound that inhibits acute inflammatory IL-1β gene transcription regulated by IL-1β eRNA of claim 9, wherein the gapmer compound that is complementary to Region C is selected from the group consisting of SEQ ID NOs. 25, 51, 121, 122, and combinations thereof.
